(19) United States
(12) Patent Application Publication
(10) Pub. No.: US 2007/0155744 A1
(43) Pub. Date: Jul. 5, 2007

(54) N-benzyl-3,4-dihyroxypyridine-2-carboxamide and n-benzyl-2,3-dihydroxypyridine-4- carboxamide compounds useful as hiv integrase inhibitors

(75) Inventors: Philip Jones, Pomezia (IT); Peter D. Williams, Harleysville, PA (US); Matthew M. Morrissette, Radnor, PA (US); Michelle Sparks Kuo, Gwynedd Valley, PA (US); Joseph P. Vacca, Telford, PA (US)

Correspondence Address: MERCK AND CO., INC, P O BOX 2000, RAHWAY, NJ, 07065-0907, US

(73) Assignees: ISTITUTO DI RICERCHE DI BIOLOGIA MOLECOLARE P, ANGELETTI S.P.A., ROMA (IT); MERCK & CO., INC., RAHWAY, NJ (US)

(21) Appl. No.: 10/587,330

(22) PCT Filed: Jan. 26, 2005
(86) PCT No.: PCT/US05/02472

§ 371 Date: Jul. 27, 2006

Related U.S. Application Data
(60) Provisional application No. 60/540,538, filed on Jan. 30, 2004.

(51) Int. Cl. A61K 31/5377; A61K 31/513; A61K 31/497; A61K 31/496; A61K 31/4545; A61K 31/444; C07D 413/14; C07D 403/14; C07D 401/14

(52) U.S. Cl. 514/235.2; 514/332; 514/341; 514/346; 514/342; 514/318; 514/303; 514/253.12; 514/255.05; 544/128; 544/405; 544/360; 546/193; 546/261; 514/269; 544/310

(57) ABSTRACT

N-Benzyl-dihydroxypyridine carboxamide compounds are inhibitors of HIV integrase and inhibitors of HIV replication. In one embodiment, the dihydroxypyridine carboxamides are of Formula (I) wherein Q is Formula (II) or Formula (III); T is Formula (IV); and R¹, R², X¹,X²,X³, and Y₁ are defined herein. The compounds are useful in the prevention and treatment of infection by HIV and in the prevention, delay in the onset, and treatment of AIDS. The compounds are employed against HIV infection and AIDS as compounds per se or in the form of pharmaceutically acceptable salts. The compounds and their salts can be employed as ingredients in pharmaceutical compositions, optionally in combination with other antivirals, immunomodulators, antibiotics or vaccines.

### FIELD OF THE INVENTION

The present invention is directed to N-benzyl-dihydroxypyridine carboxamide compounds, and pharmaceutically acceptable salts thereof, their synthesis, and their use as inhibitors of the HIV integrase enzyme. The compounds and pharmaceutically acceptable salts thereof of the present invention are useful for preventing or treating infection by HIV and for preventing, treating or delaying the onset of AIDS.

### BACKGROUND OF THE INVENTION

A retrovirus designated human immunodeficiency virus (HIV) is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the insertion by virally-encoded integrase of proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)]. Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells. The particular advantage of the present invention is highly specific inhibition of HIV integrase and HIV replication.

The following references are of interest as background:

U.S. Pat. No. 6,380,249, U.S. Pat. No. 6,306,891, and U.S. Pat. No. 6,262,055 disclose 2,4-dioxobutyric acids and acid esters useful as HIV integrase inhibitors.

WO 01/00578 discloses 1-(aromatic- or heteroaromatic-substituted)-3-(heteroaromatic substituted)-1,3-propanediones useful as HIV integrase inhibitors.

US 2003/0055071 (corresponding to WO 02/30930), WO 02/30426, and WO 02/55079 each disclose certain 8-hydroxy-1,6-naphthyridine-7-carboxamides as HIV integrase inhibitors.

WO 02/036734 discloses certain aza- and polyaza-naphthalenyl ketones to be HIV integrase inhibitors.

WO 03/016275 (to which EP 1422218 corresponds) discloses certain compounds having integrase inhibitory activity.

WO 03/35076 discloses certain 5,6-dihydroxypyrimidine-4-carboxamides as HIV integrase inhibitors, and WO 03/35077 discloses certain N-substituted 5-hydroxy-6-oxo-1,6-dihydropyrimidine-4-carboxamides as HIV integrase inhibitors.

WO 03/062204 discloses certain hydroxynaphthyridinone carboxamides that are useful as IV integrase inhibitors.

WO 04/004657 discloses certain hydroxypyrrole derivatives that are HIV integrase inhibitors. WO 2004/062613 discloses certain pyrimidine carboxamides as HIV integrase inhibitors.

### SUMMARY OF THE INVENTION

The present invention is directed to N-benzyl-dihydroxypyridine carboxamides. These compounds are useful in the inhibition of HIV integrase, the prevention of infection by HIV, the treatment of infection by HIV and in the prevention, treatment, and delay in the onset of AIDS and/or ARC, either as compounds or their pharmaceutically acceptable salts or hydrates (when appropriate), or as pharmaceutical composition ingredients, whether or not in combination with other HIV/AIDS antivirals, anti-infectives, immunomodulators, antibiotics or vaccines. More particularly, the present invention includes compounds of Formula J, and pharmaceutically acceptable salts thereof: 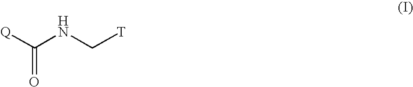
wherein:
Q is: 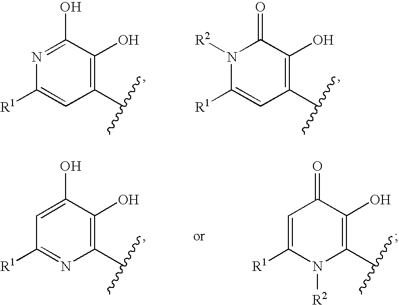
T is: 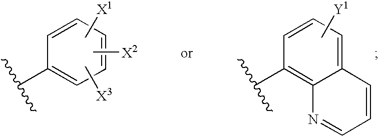
X¹, X² and X³ are each independently selected from the group consisting of —H, halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, and HetA
Y¹ is —H, halo, —C₁₋₄ alkyl, or —C₁₋₄ fluoroalkyl;
R¹ is:

(1) —H

(2) —C₁₋₆ alkyl,

(3) —C₁₋₆ fluoroalkyl,

(4) —C₁₋₆ alkyl-N(R^{a})R^{b},

(5) —C₁₋₆ alkyl-N(R^{a})—C(═O)—R^{b},

(6) —C(═O)—R^{a},

(7) —C(═O)OR^{a},

(8) —C(═O)—N(R^{a})R^{b},

(9) —C(═O)—N(R^{a})—C₁₋₆ alkyl-aryl,

(10) —HetB,

(11) —C(═O)—N(R^{a})—C₁₋₆ alkyl—HetB,

(12) —C₁₋₆ alkyl-HetC,

(13) —C(═O)—HetC,

(14) —C(═O)-aryl, or

(15) —C(═O)—HetB;

each HetA is independently a 5- or 6-membered heteroaromatic ring containing from 1 to 4 heteroatoms independently selected from N, O and S, wherein the heteroaromatic ring is optionally substituted with 1 or 2 substituents each of which is independently a —C₁₋₄ alkyl;

HetB is:

(A) a 5- or 6-membered heteroaromatic ring containing from 1 to 4 heteroatoms independently selected from N, O and S; wherein the heteroaromatic ring is attached to the rest of the compound via a carbon atom in the ring, and wherein the heteroaromatic ring is:
   (i) optionally substituted with 1 or 2 substituents each of which is independently a —C₁₋₄ alkyl; and
   (ii) optionally substituted with aryl or —C₁₋₄ alkyl-aryl; or
(B) a 9- or 10-membered aromatic heterobicyclic fused ring system containing from 1 to 4 heteroatoms independently selected from N, O and S; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
   (i) optionally substituted with 1 or 2 substituents each of which is independently a —C₁₋₄ alkyl; and
   (ii) optionally substituted with aryl or —C₁₋₄ alkyl-aryl;
      HetC is a 4- to 7-membered saturated heterocyclic ring containing at least one carbon atom and a total of from 1 to 4 heteroatoms independently selected from 1 to 4 N atoms, from 0 to 2 O atoms, and from 0 to 2 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with 1 or 2 substituents each of which is independently a —C₁₋₄ alkyl, —C₁₋₄ alkyl-N(R^{a})R^{b}, or —C(═O)OR^{a}; and
(ii) optionally substituted with aryl, —C₁₋₄ alkyl-aryl, HetD, or —C₁₋₄ alkyl-HetD; wherein HetD is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S;
   R² is —C₁₋₆ alkyl or —C₁₋₆ alkyl-aryl;
   aryl is phenyl or naphthyl;
   each R^{a} is independently H or C₁₋₆ alkyl; and
   each R^{b} is independently H or C₁₋₆ alkyl.

The present invention also includes pharmaceutical compositions containing a compound of the present invention and methods of preparing such pharmaceutical compositions. The present invention further includes methods of treating AIDS, methods of delaying the onset of AIDS, methods of preventing AIDS, methods of preventing infection by HIV, and methods of treating infection by HIV.

Other embodiments, aspects and features of the present invention are either further described in or will be apparent from the ensuing description, examples and appended claims.

### DETAILED DESCRIPTION OF THE INVENTION

The present invention includes compounds of Formula I above, and pharmaceutically acceptable salts thereof. These compounds and pharmaceutically acceptable salts thereof are HIV integrase inhibitors.

A first embodiment of the present invention is a compound of Formula I, or a pharmaceutically acceptable salt thereof, wherein R¹ is (1) —C₁₋₆ fluoroalkyl containing at least one CF₃ group, (2) —C(═O)—R^{a}, (3) —C(═O)OR^{a}, (4) —C(═O)—N(R^{a})R^{b}, (5) —C(═O)—N(R^{a})—C₁₋₆ alkyl-aryl, (6) —C(═O)—N(R^{a})—C₁₋₆ alkyl—HetB, or (7) —C(═O)—HetC; and all other variables are as originally defined (i.e., as defined in the Summary of the Invention). This embodiment is based on the discovery that the presence of an electron withdrawing group (e.g., groups (1) to (7) above) in the 6-position of a pyridine 2-carboxamide or in the 2-position of a pyridine 4-carboxamide results in increased integrase inhibition activity relative to no substitution or substitution with an electron donating group. In an aspect of this embodiment, the electron withdrawing group is in the 6-position of a pyridine 2-carboxamide.

A second embodiment of the present invention is a compound of Formula I, or a pharmaceutically acceptable salt thereof, wherein R¹ is:

(1) —H,

(2) —C₁₋₃ alkyl,

(3) —C₁₋₃ fluoroalkyl,

(4) —C₁₋₃ alkyl-NH₂,

(5) —C₁₋₃ alkyl-NH(—C₁₋₃ alkyl),

(6) —C₁₋₃ alkyl-N(—C₁₋₃ alkyl)₂,

(7) —C₁₋₃ alkyl-NH—C(═O)—C₁₋₃ alkyl,

(8) —C₁₋₃ alkyl-N(—C₁₋₃ alkyl)-C(═O)—C₁₋₃ alkyl,

(9) —C(═O)H,

(10) —C(═O)—C₁₋₃ alkyl,

(11) —CO₂H,

(12) —C(═O)O—C₁₋₃ alkyl,

(13) —C(═O)—NH(—C₁₋₃ alkyl),

(14) —C(═O)—N(—C₁₋₃ alkyl)₂,

(15) —C(═O)—NH—CH₂-phenyl,

(16) —C(═O)—N(CH₃)—CH₂-phenyl,

(17) —HetB,

(18) —C(═O)—NH—CH₂—HetB,

(19) —C(═O)—N(CH₃)—CH₂—HetB,

(20) —CH₂—HetC,

(21) —CH(CH₃)—HetC, or

(22) —C(═O)—HetC;

and all other variables are as originally defined.

A third embodiment of the present invention is a compound of Formula I, or a pharmaceutically acceptable salt thereof, wherein R¹ is:

(1) —C₁₋₃ fluoroalkyl containing at least one CF₃,

(2) —C₁₋₃ alkyl-N(—C₁₋₃ alkyl)₂,

(3) —C(═O)—C₁₋₃ alkyl,

(4) —CO₂H,

(5) —C(═O)O—C₁₋₃ alkyl,

(6) —C(═O)—NH(—C₁₋₃ alkyl),

(7) —C(═O)—N(—C₁₋₃ alkyl)₂,

(8) —C(═O)—NH—CH₂-phenyl,

(9) —C(═O)—N(CH₃)—CH₂-phenyl,

(10) —HetB,

(11) —C(═O)—NH—CH₂—HetB,

(12) —C(═O)—N(CH₃)—CH₂—HetB, or

(13) —C(═O)—HetC;

and all other variables are as originally defined above. In an aspect of the third embodiment, R¹ is any one of the above groups (1) and (3) to (13) (i.e., the definition of R¹ excludes (2) —C₁₋₃ alkyl-N(—C₁₋₃ alkyl)₂).

A fourth embodiment of the present invention is a compound of Formula I, or a pharmaceutically acceptable salt thereof, wherein R¹ is:

(1) —CF₃,

(2) —CH(CH₃)—N(CH₃)₂,

(3) —C(═O)—CH₃,

(4) —CO₂H,

(5) —C(═O)OCH₃,

(6) —C(═O)—NH(CH₃),

(7) —C(═O)—N(CH₃)₂,

(8) —C(═O)—NH(CH₂CH₃),

(9) —C(═O)—N(CH₂CH₃)₂,

(10) —C(═O)—NH(CH(CH₃)₂),

(11) —C(═O)—NH—CH₂-phenyl,

(12) —C(═O)—N(CH₃)—CH₂-phenyl,

(13) —HetB,

(14) —C(═O)—NH—CH₂—HetB,

(15) —C(═O)—N(CH₃)—CH₂—HetB, or

(16) —C(═O)—HetC;

and all other variables are as originally defined above. In an aspect of the fourth embodiment, R¹ is any one of the above groups (1) and (3) to (16) (i.e., the definition of R¹ excludes (2) —CH(CH₃)—N(CH₃)₂).

A fifth embodiment of the present invention is a compound of Formula I, or a pharmaceutically acceptable salt thereof, wherein HetB is:
(A) a 5- or 6-membered heteroaromatic ring containing a total of from 1 to 3 heteroatoms independently selected from zero to 3 N atoms, zero or 1 O atoms, and zero or 1 S atoms; wherein the heteroaromatic ring is attached to the rest of the compound via a carbon atom in the ring, and wherein the heteroaromatic ring is:
   (i) optionally substituted with 1 or 2 substituents each of which is independently a —C₁₋₃ alkyl; and
   (ii) optionally substituted with phenyl or —CH₂-phenyl; or
(B) a 9- or 10-membered aromatic heterobicyclic fused ring system containing a total of from 1 to 4 heteroatoms independently selected from 1 to 4 N atoms, zero or 1 O atoms, and zero or 1 S atoms; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
   (i) optionally substituted with 1 or 2 substituents each of which is independently a —C₁₋₃ alkyl; and
   (ii) optionally substituted with phenyl or —CH₂-phenyl;
      and all other variables are as originally defined or as defined in any one of the preceding embodiments.

A sixth embodiment of the present invention is a compound of Formula I, or a pharmaceutically acceptable salt thereof, wherein HetB is a heteroaromatic ring selected from the group consisting of oxadiazolyl, thiophenyl (alternatively referred to in the art as “thienyl”), pyrazolyl, thiazolyl, isothiazolyl, oxazolyl, isoxazolyl, imidazolyl, pyridinyl, pyrimidinyl, pyrazinyl, and pyridoimidazolyl; wherein the heteroaromatic ring is attached to the rest of the compound via a carbon atom in the ring, and wherein the heteroaromatic ring is optionally substituted with methyl or phenyl;

and all other variables are as originally defined or as defined in any one of the first four embodiments.

A seventh embodiment of the present invention is a compound of Formula I, or a pharmaceutically acceptable salt thereof, wherein HetC is a 5- or 6-membered saturated heterocyclic ring containing a total of from 1 to 3 heteroatoms independently selected from 1 to 3 N atoms, zero or 1 O atoms, and zero or 1 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with —C₁₋₃ alkyl, —(CH₂)₁₋₂—NH(—C₁₋₃ alkyl), —(CH₂)₁₋₂—N(—C₁₋₃ alkyl)₂ or —C(═O)O—C₁₋₃ alkyl; and
(ii) optionally substituted with phenyl, —CH₂-phenyl, HetD, or —(CH₂)₁₋₂—HetD; wherein HetD is (i) a 5- or 6-membered heteroaromatic ring containing a total of from 1 to 3 heteroatoms independently selected from zero to 3 N atoms, zero or 1 O atoms, and zero or 1 S atoms or (ii) a 5- or 6-membered saturated heterocyclic ring containing a total of from 1 to 3 heteroatoms independently selected from 1 to 3 N atoms, zero or 1 O atoms, and zero or 1 S atoms;
   and all other variables are as originally defined or as defined in any one of the preceding embodiments.

An eighth embodiment of the present invention is a compound of Formula I, or a pharmaceutically acceptable salt thereof, wherein HetC is a heterocyclic ring selected from the group consisting of pyrrolidinyl, morpholinyl, piperidinyl, piperazinyl, and piperidinyl fused with a benzene ring; wherein the heterocyclic ring is attached to the rest of the compound via a N atom in the ring, and wherein the heterocyclic ring is optionally substituted with methyl, —CH₂N(CH₃)₂, —C(═O)OCH₂CH₃, pyridinyl, —CH₂-pyridinyl, —CH₂-morpholinyl, or —CH₂CH₂-morpholinyl; and all other variables are as originally defined or as defined in any one of the first six embodiments.

An ninth embodiment of the present invention is a compound of Formula I, or a pharmaceutically acceptable salt thereof, wherein T is: 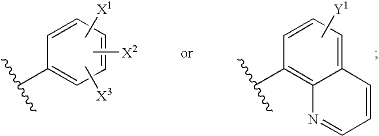
X¹ is fluoro, chloro, methyl, trifluoromethyl, methoxy, —SO₂CH₃, —C(═O)—NH(CH₃), —C(═O)—N(CH₃)₂, or oxadiazolyl;
X² and X³ are each independently selected from the group consisting of —H, fluoro, chloro, methyl, trifluoromethyl, methoxy, —SO₂CH₃, —C(═O)—NH(CH₃), and —C(═O)—N(CH₃)₂;
Y¹ is —H, fluoro, chloro, methyl, or trifluoromethyl;
and all other variables are as originally defined or as defined in any one of the preceding embodiments.

A tenth embodiment of the present invention is a compound of Formula I, or a pharmaceutically acceptable salt thereof, wherein T is 4-fluorophenyl; and all other variables are as originally defined or as defined in any one of the first eight embodiments.

An eleventh embodiment of the present invention is a compound of Formula I, or a pharmaceutically acceptable salt thereof, wherein R² is —C₁₋₃ alkyl or —CH₂-phenyl; and all other variables are as originally defined or as defined in any one of the preceding embodiments.

A twelfth embodiment of the present invention is a compound of Formula I, or a pharmaceutically acceptable salt thereof, wherein R² is methyl; and all other variables are as originally defined or as defined in any one of the first ten embodiments.

A thirteenth embodiment of the present invention is a compound of Formula I, wherein each R^{a} and R^{b} is independently H or C₁₋₄ alkyl; and all other variables are as originally defined or as defined in any one of the preceding embodiments.

A fourteenth embodiment of the present invention is a compound of Formula I, wherein each R^{a} and R^{b} is independently H or methyl; and all other variables are as originally defined or as defined in any one of the first twelve embodiments.

A first class of the present invention includes compounds of Formula II, and pharmaceutically acceptable salts thereof: 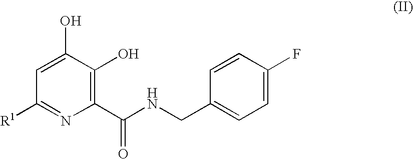
wherein R¹ is:

(1) —C₁₋₄ fluoroalkyl,

(2) —C₁₋₄ alkyl-N(R^{a})R^{b},

(3) —C(═O)—R^{a},

(4) —C(═O)OR^{a},

(5) —C(═O)—N(R^{a})R^{b},

(6) —C(═O)—N(R^{a})—C₁₋₄ alkyl-aryl,

(7) —HetB,

(8) —C(═O)—N(R^{a})—C₁₋₄ allyl—HetB, or

(9) —C(═O)—HetC;

HetB and HetC are each as originally defined above;

aryl is phenyl or naphthyl;

each R^{a} is independently H or C₁₋₄ alkyl; and

each R^{b} is independently H or C₁₋₄ alkyl.

A sub-class of the first class includes compounds of Formula II, and pharmaceutically acceptable salts thereof, wherein R¹ is any one of groups (1) and (3) to (9) (i.e., the definition of R¹ excludes (2) —C₁₋₄ alkyl-N(R^{a})R^{b}); and all other variables are as defined in the first class.

Another sub-class of the first class includes compounds of Formula II, and pharmaceutically acceptable salts thereof, wherein R¹ is:

(1) —C₁₋₃ fluoroalkyl,

(2) —C₁₋₃ alkyl-N(—C₁₋₃ alkyl)₂,

(3) —C(═O)—C₁₋₃ alkyl,

(4) —CO₂H,

(5) —C(═O)O—C₁₋₃ alkyl,

(6) —C(═O)—NH(—C₁₋₃ alkyl),

(7) —C(═O)—N(—C₁₋₃ alkyl)₂,

(8) —C(═O)—NH—CH₂-phenyl,

(9) —C(═O)—N(CH₃)—CH₂-phenyl,

(10) —HetB,

(11) —C(═O)—NH—CH₂—HetB,

(12) —C(═O)—N(CH₃)—CH₂—HetB, or

(13) —C(═O)—HetC;

HetB is as defined in the fifth embodiment; HetC is as defined in the seventh embodiment; and all other variables are as defined above in the first class. In a feature of this sub-class, R¹ is any one of groups (1) and (3) to (13) (i.e., the definition of R¹ excludes (2) —C₁₋₃ alkyl-N(—C₁₋₃ alkyl)₂).

Still another sub-class of the first class includes compounds of Formula II, and pharmaceutically acceptable salts thereof, wherein R¹ is:

(1) —CF₃,

(2) —C(═O)—CH₃,

(3) —CO₂H,

(4) —C(═O)OCH₃,

(5) —C(═O)—NH(CH₃),

(6) —C(═O)—N(CH₃)₂,

(7) —C(═O)—NH(CH₂CH₃),

(8) —C(═O)—N(CH₂CH₃)₂,

(9) —C(═O)—NH(CH(CH₃)₂),

(10) —C(═O)—NH—CH₂-phenyl,

(11) —C(═O)—N(CH₃)—CH₂-phenyl,

(12) —HetB,

(13) —C(═O)—NH—CH₂—HetB,

(14) —C(═O)—N(CH₃)—CH₂—HetB, or

(15) —C(═O)—HetC;

and all other variables are as defined in the first class or in the preceding sub-class.

Still another sub-class of the first class includes compounds of Formula II, and pharmaceutically acceptable salts thereof, wherein R¹ is as defined in the preceding sub-class except that in addition to groups (1) to (15) R¹ can also be —CH(CH₃)—N(CH₃)₂; and all other variables are as defined in the preceding sub-class.

A second class of the present invention includes compounds of Formula III, and pharmaceutically acceptable salts thereof: 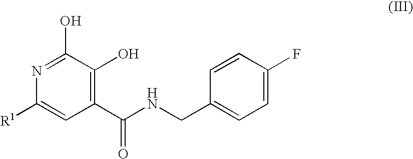
wherein:
R¹ is:

(1) —C₁₋₄ fluoroalkyl,

(2) —C₁₋₄ alkyl-N(R^{a})—C(═O)—R^{b},

(3) —C(═O)—R^{a},

(4) —C(═O)OR^{a},

(5) —C(═O)—N(R^{a})R^{b},

(6) —C(═O)—N(R^{a})—C₁₋₁₄ alkyl-aryl,

(7) —HetB,

(8) —C(═O)—N(R^{a})—C₁₋₄ alkyl—HetB,

(9) —C₁₋₄ alkyl-HetC, or

(10) —C(═O)—HetC;

HetB and HetC are each as originally defined above;

aryl is phenyl or naphthyl;

R^{a} is H or C₁₋₄ alkyl; and

R^{b} is H or C₁₋₄ alkyl.

A sub-class of the second class includes compounds of Formula III, and pharmaceutically acceptable salts thereof, wherein R¹ is any one of groups (1), (3) to (8) and (10) (i.e., the definition of R¹ excludes (2) —C₁₋₄ alkyl-N(R^{a})—C(═O)—R^{b} and (9)-C₁₋₄ alkyl-HetC); and all other variables are as defined in the second class.

Another sub-class of the second class includes compounds of Formula III, and pharmaceutically acceptable salts thereof, wherein R¹ is:

(1) —C₁₋₃ fluoroalkyl,

(2) —C₁₋₃ alkyl-N(—C₁₋₃ alkyl)-C(═O)—C₁₋₃ alkyl,

(3) —C(═O)—C₁₋₃ alkyl,

(4) —CO₂H,

(5) —C(═O)O—C₁₋₃ alkyl,

(6) —C(═O)—NH(—C₁₋₃ alkyl),

(7) —C(═O)—N(—C₁₋₃ alkyl)₂,

(8) —C(═O)—NH—CH₂-phenyl,

(9) —C(═O)—N(CH₃)—CH₂-phenyl,

(10) —HetB,

(11) —C(═O)—NH—CH₂—HetB,

(12) —C(═O)—N(CH₃)—CH₂—HetB,

(13) —CH₂—HetC,

(14) —CH(CH₃)—HetC, or

(15) —C(═O)—HetC;

HetB is as defined in the fifth embodiment; HetC is as defined in the seventh embodiment; and all other variables are as defined above in the second class. In a feature of this sub-class, R¹ is any one of groups (1), (3) to (12), and (15) (i.e., the definition of R¹ excludes (2) —C₁₋₃ alkyl-N(—C₁₋₃ alkyl)-C(═O)—C₁₋₃ alkyl, (13) —CH₂—HetC, and (14) —CH(CH₃)—HetC).

Still another sub-class of the second class includes compounds of Formula III, and pharmaceutically acceptable salts thereof, wherein R¹ is:

(1) —CF₃,

(2) —C(═O)—CH₃,

(3) —CO₂H,

(4) —C(═O)OCH₃,

(5) —C(═O)—NH(CH₃),

(6) —C(═O)—N(CH₃)₂,

(7) —C(═O)—NH(CH₂CH₃),

(8) —C(═O)—N(CH₂CH₃)₂,

(9) —C(═O)—NH(CH(CH₃)₂),

(10) —C(═O)—NH—CH₂-phenyl,

(11) —C(═O)—N(CH₃)—CH₂-phenyl,

(12) —HetB,

(13) —C(═O)—NH—CH₂—HetB,

(14) —C(═O)—N(CH₃)—CH₂—HetB, or

(15) —C(═O)—HetC;

and all other variables are as defined in the second class or in the preceding sub-class.

Still another sub-class of the second class includes compounds of Formula III, and pharmaceutically acceptable salts thereof, wherein R¹ is as defined in the preceding sub-class except that in addition to groups (1) to (15) R¹ can also be —CH(CH₃)—N(CH₃)—C(═O)CH₃, —CH₂—HetC, or —CH(CH₃)—HetC; and all other variables are as defined in the preceding sub-class.

A third class of the present invention includes compounds of Formula IV, and pharmaceutically acceptable salts thereof: 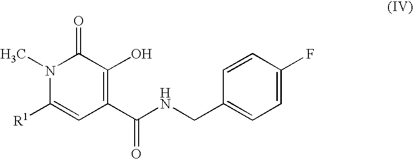
wherein R¹ is:

(1) —H,

(2) —C₁₋₄ alkyl,

(3) —C₁₋₄ fluoroalkyl,

(4) —C(═O)—R^{a},

(5) —C(═O)OR^{a},

(6) —C(═O)—N(R^{a})R^{b},

(7) —C(═O)—N(R^{a})—C₁₋₄ alkyl-aryl,

(8) —HetB,

(9) —C(═O)—N(R^{a})—C₁₋₄ alkyl—HetB, or

(10) —C(═O)—HetC;

HetB and HetC are each as originally defined above;

aryl is phenyl or naphthyl;

R^{a} is H or C₁₋₄ alkyl; and

R^{b} is H or C₁₋₄ alkyl.

A sub-class of the third class includes compounds of Formula IV, and pharmaceutically acceptable salts thereof, wherein R¹ is any one of groups (3) to (10) (i.e., the definition of R¹ excludes (1) —H and (2) —C₁₋₄ alkyl); and all other variables are as defined in the third class.

Another sub-class of the third class includes compounds of Formula IV, and pharmaceutically acceptable salts thereof, wherein R¹ is:

(1) —H,

(2) —C₁₋₃ alkyl,

(3) —C₁₋₃ fluoroalkyl,

(4) —C(═O)—C₁₋₃ alkyl,

(5) —CO₂H,

(6) —C(═O)O—C₁₋₃ alkyl,

(7) —C(═O)—NH(—C₁₋₃ alkyl),

(8) —C(═O)—N(—C₁₋₃ alkyl)₂,

(9) —C(═O)—NH—CH₂-phenyl,

(10) —C(═O)—N(CH₃)—CH₂-phenyl,

(11) —HetB,

(12) —C(═O)—NH—CH₂—HetB,

(13) —C(═O)—N(CH₃)—CH₂—HetB, or

(14) —C(═O)—HetC;

HetB is as defined in the fifth embodiment; HetC is as defined in the seventh embodiment; and all other variables are as defined above in the third class. In a feature of this sub-class, R¹ is any one of groups (3) to (14) (i.e., the definition of R¹ excludes (1) —H and (2) —C₁₋₃ alkyl).

Still another sub-class of the third class includes compounds of Formula IV, and pharmaceutically acceptable salts thereof, wherein R¹ is:

(1) —CF₃,

(2) —C(═O)—CH₃,

(3) —CO₂H,

(4) —C(═O)OCH₃,

(5) —C(═O)—NH(CH₃),

(6) —C(═O)—N(CH₃)₂,

(7) —C(═O)—NH(CH₂CH₃),

(8) —C(═O)—N(CH₂CH₃)₂,

(9) —C(═O)—NH(CH(CH₃)₂),

(10) —C(═O)—NH—CH₂-phenyl,

(11) —C(═O)—N(CH₃)—CH₂-phenyl,

(12) —HetB,

(13) —C(═O)—NH—CH₂—HetB,

(14) —C(═O)—N(CH₃)—CH₂—HetB, or

(15) —C(═O)—HetC.

and all other variables are as defined in the third class or in the preceding sub-class.

Still another sub-class of the third class includes compounds of Formula III, and pharmaceutically acceptable salts thereof, wherein R¹ is as defined in the preceding sub-class except that in addition to groups (1) to (15) R¹ can also be —H or methyl; and all other variables are as defined in the preceding sub-class.

A fifteenth embodiment of the present invention is a compound, or a pharmaceutically acceptable salt thereof, selected from the group consisting of the compounds set forth in Table 1 below.

Other embodiments of the present invention include the following:

(a) A pharmaceutical composition comprising an effective amount of a compound of Formula (I) and a pharmaceutically acceptable carrier.

(b) A pharmaceutical composition which comprises the product prepared by combining (e.g., mixing) an effective amount of a compound of Formula (I) and a pharmaceutically acceptable carrier.

(c) The pharmaceutical composition of (a) or (b), further comprising an effective amount of an HIV infection/AIDS treatment agent selected from the group consisting of HIV/AIDS antiviral agents, immunomodulators, and anti-infective agents.

(d) The pharmaceutical composition of (c), wherein the HIV infection/AIDS treatment agent is an antiviral selected from the group consisting of IV protease inhibitors, non-nucleoside HIV reverse transcriptase inhibitors, and nucleoside HIV reverse transcriptase inhibitors.

(e) A pharmaceutical combination which is (i) a compound of Formula I and (ii) an HIV infection/AIDS treatment agent selected from the group consisting of HIV/ADS antiviral agents, immunomodulators, and anti-infective agents; wherein the compound of Formula I and the HIV infection/AIDS treatment agent are each employed in an amount that renders the combination effective for inhibiting HIV integrase, for treating or preventing infection by HIV, or for preventing, treating or delaying the onset of AIDS.

(f) The combination of (e), wherein the HIV infection/AIDS treatment agent is an antiviral selected from the group consisting of HIV protease inhibitors, non-nucleoside HIV reverse transcriptase inhibitors and nucleoside HIV reverse transcriptase inhibitors.

(g) A method of inhibiting HIV integrase in a subject in need thereof which comprises administering to the subject an effective amount of a compound of Formula I.

(h) A method of preventing or treating infection by HIV in a subject in need thereof which comprises administering to the subject an effective amount of a compound of Formula I.

(i) The method of (h), wherein the compound of Formula (I) is administered in combination with an effective amount of at least one antiviral selected from the group consisting of HIV protease inhibitors, non-nucleoside HIV reverse transcriptase inhibitors, and nucleoside HIV reverse transcriptase inhibitors.

(j) A method of preventing, treating or delaying the onset of AIDS in a subject in need thereof which comprises administering to the subject an effective amount of a compound of Formula I.

(k) The method of (j), wherein the compound is administered in combination with an effective amount of at least one antiviral selected from the group consisting of HIV protease inhibitors, non-nucleoside HIV reverse transcriptase inhibitors, and nucleoside HIV reverse transcriptase inhibitors

(l) A method of inhibiting HIV integrase in a subject in need thereof which comprises administering to the subject the pharmaceutical composition of (a), (b), (c) or (d) or the combination of (e) or (f).

(m) A method of preventing or treating infection by HIV in a subject in need thereof which comprises administering to the subject the pharmaceutical composition of (a), (b), (c) or (d) or the combination of (e) or (f).

(n) A method of preventing, treating or delaying the onset of AIDS in a subject in need thereof which comprises administering to the subject the pharmaceutical composition of (a), (b), (c) or (d) or the combination of (e) or (f).

The present invention also includes a compound of the present invention (i) for use in, (ii) for use as a medicament for, or (iii) for use in the preparation of a medicament for: (a) inhibiting HIV integrase, (b) preventing or treating infection by HIV, or (c) preventing, treating or delaying the onset of AIDS. In these uses, the compounds of the present invention can optionally be employed in combination with one or more HIV/AIDS treatment agents selected from HIV/AIDS antiviral agents, anti-infective agents, and immunomodulators.

Additional embodiments of the invention include the pharmaceutical compositions, combinations and methods set forth in (a)-(n) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl.

The term “-alkyl-” refers to any linear or branched chain alkylene (or alternatively “alkanediyl”) having a number of carbon atoms in the specified range. Thus, for example, if “—C₁₋₆ alkyl-” refers to a C₁ to C₆ linear or branched alkylenes. A class of alkylenes of particular interest with respect to the invention is —(CH₂)₁₋₆—, and sub-classes of particular interest include —(CH₂)₁₋₄—, —(CH₂)₁₋₃—, —(CH₂)₁₋₂—, and —CH₂—. Also of interest is the alkylene —CH(CH₃)—.

The term “halogen” (or “halo”) refers to fluorine, chlorine, bromine and iodine (alternatively referred to as fluoro, chloro, bromo, and iodo).

The term “fluoroalkyl” refers to an alkyl group as defined above in which one or more of the hydrogen atoms has been replaced with a fluorine. Thus, for example, “C₁₋₄ fluoroalkyl” (or “C₁-C₄ fluoroalkyl”) refers to a C₁ to C₄ linear or branched alkyl group as defined above with one or more fluorine substituents. Particularly suitable fluoroalkyl groups are those containing at least one trifluoromethyl group, such as those in the series (CH₂)₀₋₃CF₃ (e.g., trifluoromethyl, 2,2,2-trifluoroethyl, and 3,3,3-trifluoro-n-propyl).

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

When any variable (e.g., R^{a} or R^{b}) occurs more than one time in any constituent or in Formula I or in any other formula depicting and describing compounds of the invention, its definition on each occurrence is independent of its definition at every other occurrence. Also, combinations of substituents and/or variables are permissible only if such combinations result in stable compounds.

The term “substituted” (e.g., as in “is optionally substituted with from 1 to 5 substituents . . . ”) includes mono- and poly-substitution by a named substituent to the extent such single and multiple substitution (including multiple substitution at the same site) is chemically allowed. Unless expressly stated to the contrary, substitution by a named substituent is permitted on any atom in a ring (e.g., aryl, a heteroaromatic ring, or a saturated heterocyclic ring) provided such ring substitution is chemically allowed and results in a stable compound.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject).

The symbol “ [symbol] ” in front of an open bond in the structural formula of a group marks the point of attachment of the group to the rest of the molecule.

When a compound of the present invention has one or more asymmetric centers and thus can occur as an optical isomer (e.g., an enantiomer or a diastereomer), it is understood that the present invention includes all isomeric forms of the compound, singly and in mixtures.

As would be recognized by one of ordinary skill in the art, certain of the compounds of the present invention can exist as tautomers, such as the following:
Group 1— 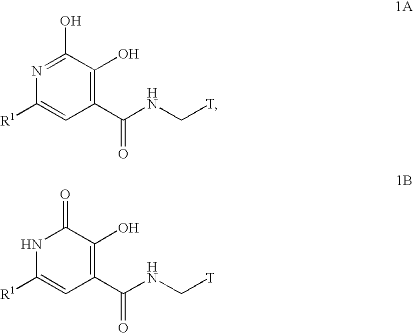
Group 2— 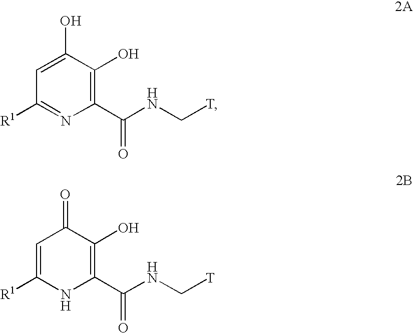
For the purposes of the present invention, a reference herein to a compound of Formula I (or II, III or IV) is a reference to compound I per se (or II, III, or IV), or to any one of its tautomers per se (e.g., 1A, 1B, 2A, 2B or the like)), or to mixtures of two or more of the foregoing.

The compounds of the present inventions are useful in the inhibition of HIV integrase, the prevention or treatment of infection by human immunodeficiency virus (HIV) and the prevention, treatment or the delay in the onset of consequent pathological conditions such as AIDS. Preventing AIDS, treating AIDS, delaying the onset of AIDS, or preventing or treating infection by BV is defined as including, but not limited to, treatment of a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

Compounds representative of the present invention have been tested for inhibition in an assay for the strand transfer activity of integrase. The assay is conducted in the manner described in WO 02/30930. Representative compounds of the present invention exhibit inhibition of strand transfer activity in this assay. For example, the compounds set forth in Table 1 below were tested in the integrase assay and demonstrated IC₅₀'s of about 5.5 micromolar or less. Further description on conducting the assay using preassembled complexes is found in Hazuda et al., J. Virol. 1997, 71: 7005-7011; Hazuda et al., Drug Design and Discovery 1997, 15: 17-24; and Hazuda et al., Science 2000, 287: 646-650.

Compounds representative of the present invention have also been tested in an assay for inhibition of acute HIV infection of T-lymphoid cells, conducted in accordance with Vacca, J. P. et al., Proc. Natl. Acad. Sci. USA 1994, 91: 4096. Representative compounds of the present invention exhibit inhibition of HIV infection in this assay. For example, the compounds set forth below in Table 1 demonstrated IC₉₅'s of less than about 20 micromolar.

The compounds of the present invention may be administered in the form of pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof). Suitable salts include acid addition salts which may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

For the purpose of inhibiting HIV integrase, preventing or treating HIV infection or preventing, treating or delaying the onset of AIDS, the compounds of the present invention may be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles.

The term “administration” and variants thereof (e.g., “administering” a compound) in reference to a compound of the invention mean providing the compound or a prodrug of the compound to the individual in need of treatment. When a compound of the invention or a prodrug thereof is provided in combination with one or more other active agents (e.g., antiviral agents useful for treating HIV infection or AIDS), “administration” and its variants are each understood to include concurrent and sequential provision of the compound or prodrug and other agents.

As used herein, the term “composition” is intended to encompass a product comprising the specified ingredients in the specified amounts, as well as any product which results, directly or indirectly, from combining the specified ingredients in the specified amounts.

By “pharmaceutically acceptable” is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of active compound sufficient to inhibit HIV integrase and thereby elicit the response being sought (i.e., an “inhibition effective amount”). When the active compound (i.e., active ingredient) is administered as the salt, references to the amount of active ingredient are to the free acid or free base form of the compound.

The pharmaceutical compositions may be in the form of orally-administrable suspensions or tablets or capsules, nasal sprays, sterile injectable preparations, for example, as sterile injectable aqueous or oleagenous suspensions or suppositories. These compositions can be prepared by methods and contain excipients which are well known in the art. Suitable methods and ingredients are described in Remington's Pharmaceutical Sciences, 18^{th} edition, edited by A. R. Gennaro, Mack Publishing Co., 1990, which is herein incorporated by reference in its entirety.

The compounds of this invention can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

As noted above, the present invention is also directed to use of the HIV integrase inhibitor compounds of the present invention with one or more agents useful in the treatment of HIV infection or AIDS. For example, the compounds of this invention may be effectively administered, whether at periods of pre-exposure and/or post-exposure, in combination with effective amounts of one or more HIV/AIDS antivirals, imunomodulators, antiinfectives, or vaccines useful for treating HIV infection or AIDS, such as those disclosed in Table 1 of WO 01/38332 or in the Table in WO 02/30930, both documents being herein incorporated by reference in their entireties. It will be understood that the scope of combinations of the compounds of this invention with HIV/AIDS antivirals, immunomodulators, anti-infectives or vaccines is not limited to the list in the above-referenced Tables in WO 01/38332 and WO 02/30930, but includes in principle any combination with any pharmaceutical composition useful for the treatment of AIDS. The HIV/AIDS antivirals and other agents will typically be employed in these combinations in their conventional dosage ranges and regimens as reported in the art, including, for example, the dosages described in the Physicians' Desk Reference, 57^{th} edition, Thomson PDR, 2003. The dosage ranges for a compound of the invention in these combinations are the same as those set forth above.

Abbreviations used in the instant specification, particularly the Schemes and Examples, include the following:
AIDS=acquired immunodeficiency syndrome
ARC=AIDS related complex
Bn=benzyl
BOP=benzotriazol-1-yloxytris-(dimethylamino)phosphonium
t-BuLi=tert-butyl lithium
DCM=dichloromethane
DMF=N,N-dimethylformamide
DMSO=dimethylsulfoxide
EDC=1-ethyl-3-(3-dimethylaminopropyl)carbodiimide
ES=electrospray
Et=ethyl
EtOH=ethanol
EtOAc=ethyl acetate
FIA-MS=flow injection analysis mass spectrometry
HIV=human immunodeficiency virus
HOBT or HOBt=1-hydroxy benzotriazole hydrate
HPLC=high performance liquid chromatography
m-CPBA=meta-chloroperbenzoic acid
Me=methyl
MeOH=methanol
MOM=methoxymethyl
NMR=nuclear magnetic resonance
Ph=phenyl
Py=pyridine
TFA=trifluoroacetic acid
THF=tetrahydrofuran
TMSCN=trimethylsilyl cyanide

The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

The compounds of the present invention can be prepared by the coupling of suitable functionalized pyridine carboxylic acids (or acid derivatives such as acid halides or esters) with the appropriate amines as shown in Scheme 1 below. The resulting product may itself be active or can then be modified by further synthetic steps to yield other compounds of the present invention.

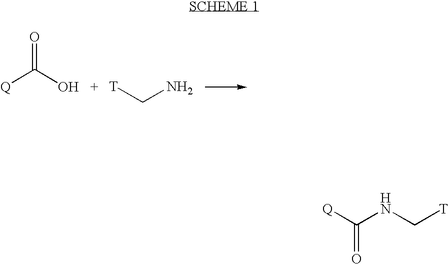

Methods for coupling carboxylic acids (and acid derivatives) with amines to form carboxamides are well known in the art. Suitable methods are described, for example, in Jerry March, Advanced Organic Chemistry, 3rd edition, John Wiley & Sons, 1985, pp. 370-376. Amines of formula T-CH₂NH₂ can be prepared using the methods described in Richard Larock, Comprehensive Organic Transformations, VCH Publishers Inc, 1989, pp 385-438, or routine variations thereof.

Schemes 2 to 10 below illustrate and expand upon the chemistry portrayed in Scheme 1. In Scheme 2 a suitably functionalized pyridine (Such as 2-0, Tetrahedron 2001, 57, 3479) can be oxidized to the corresponding N-oxide 2-1 (e.g. with m-CPBA). This pyridine can be converted to the corresponding nitrile 2.2 as described by Wilmer K. Fife J. Org. Chem. 1983, 48, 1375-1377 and Sheng-Tung Huang and Dana M. Gordon Tetrahedron Lett. 1998, 39, 9335 (e.g. with TMS-CN and Et₂NCOCl). Treatment of the nitrile with an excess of an appropriate organometallic reagents, such as a Grignard reagent, will give the corresponding ketone 2-3 after acid workup. Subsequent oxidation will give the aldehyde 2-4 and then the acid 2-5 (suitable methods are described in Jerry March, Advanced Organic Chemistry, 3rd edition, John Wiley & Sons, and Richard Larock, Comprehensive Organic Transformations, VCH Publishers Inc, 1989). Amide coupling (e.g. using PyBOP and a tertiary amine base) will form 2-6 which can be deprotected to yield 2-7 (e.g. with hydrogen and palladium on carbon as described in Theodora W. Greene and Peter G. M. Wuts, Protective Groups in Organic Synthesis, 3^{rd} Edition, Wiley-Interscience).

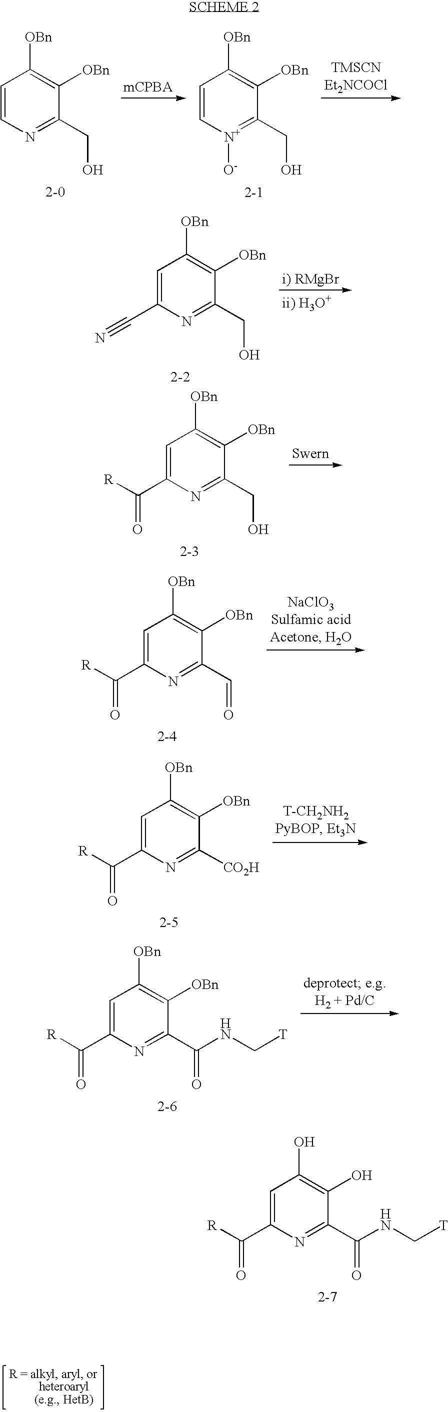

In Scheme 3 a suitably functionalized pyran (Such as 3-0, J. Med. Chem. 1988, 31, 1052) can alkylated with formaldehyde as described in Bioorg. Med. Chem. Lett. 2001, 9, 563 to give the hydroxymethyl derivative 3-1. This can be protected as under standard conditions (Theodora W. Greene and Peter G. M. Wuts, Protective Groups in Organic Synthesis, 3^{rd} Edition, Wiley-Interscience) to give the 3-benzyloxypyran 3-2. This can be oxidized as described above to give the acid 3-4. This pyran can be converted into the corresponding pyridone 3-5 by treatment with concentrated aqueous ammonia in an alcohol solvent as described in WO 01/17497. This can be doubly alkylated with benzyl bromide and K₂CO₃ to yield 3-6. Refluxing the ester with an excess of suitable amine will yield the amide 3-7. The THP-protecting group can be deprotected to yield 3-8 (e.g. with HCl in THF as described in Theodora W. Greene and Peter G. M. Wuts, Protective Groups in Organic Synthesis, 3^{rd} Edition, Wiley-Interscience). Oxidation and deprotection as described above in Scheme 2 can yield the pyridine 3-11.

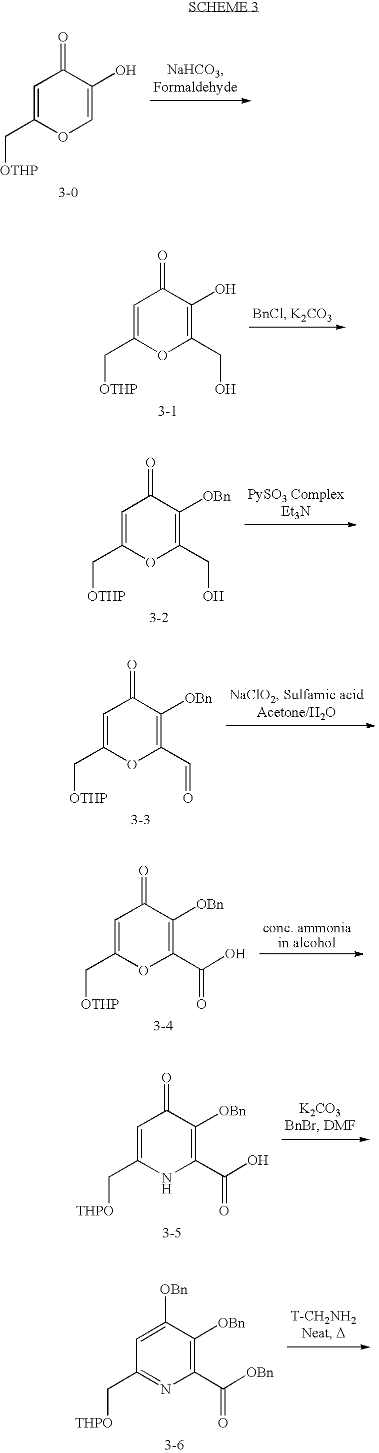

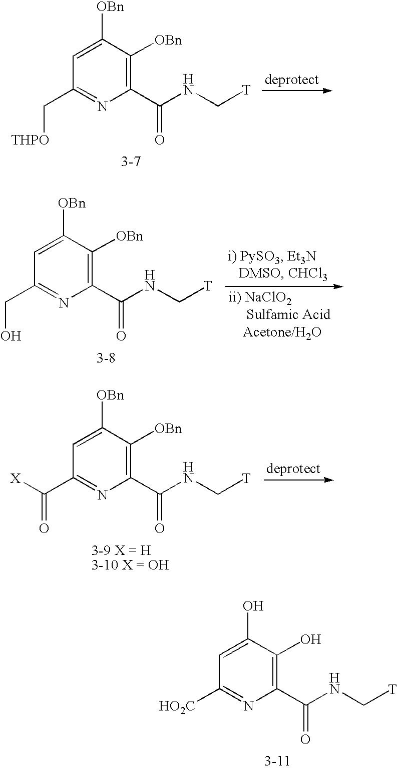

In Scheme 4 a suitably protected pyridine carboxylic acid such as 3-10 is converted into the corresponding ester as described in Jerry March, Advanced Organic Chemistry, 3^{rd} edition, John Wiley & Sons, 1985, and in Richard Larock, Comprehensive Organic Transformations, VCH Publishers Inc, 1989, (suitable methods include treatment with trimethylsilyldiazomethane or alkylation with a base and suitable organic halide. Deprotection as previously described yields the corresponding pyridine 4-2.

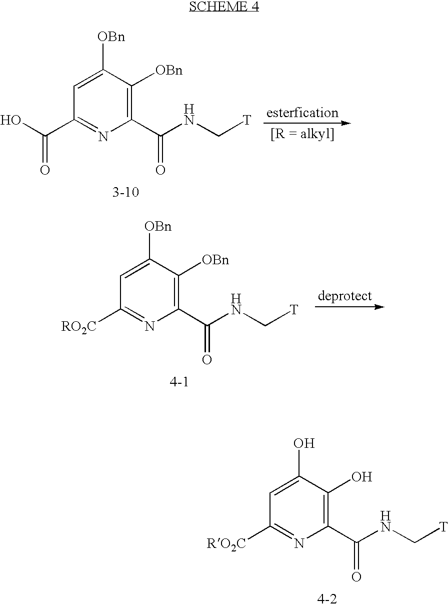

In Scheme 5, a suitably protected pyridine carboxylic acid such as 3-10 can be coupled with a variety of amines to give after deprotection the desired amide 5-1. Suitable coupling conditions include the use of BOPCl, exemplified in the scheme, and others described in Jerry March, Advanced Organic Chemistry, 3rd edition, John Wiley & Sons, 1985.

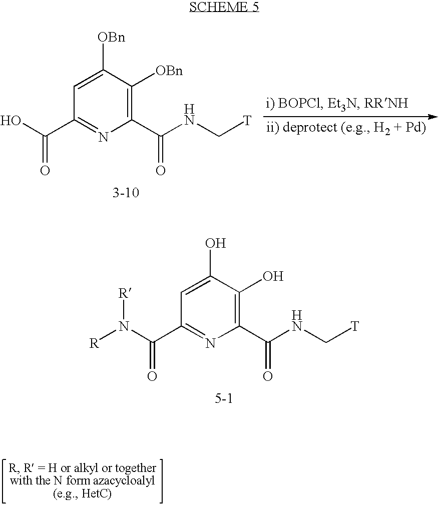

Scheme 6 illustrates how heterocyclic derivatives can be introduced at the C-6position of the pyridine to give compounds such as Compound 6B (Het=Heterocycle), wherein these heterocycles can be prepared from a starting substrate 6A containing a functional group (FG; e.g., an acid, ester, or nitrile) using methods set forth in Alan Katritzky, Comprehensive Heterocyclic Chemistry, (Pergamon Press, New York, 1984) and Comprehensive Heterocylic Chemistry II, (Pergamon Press, New York, 1996). An illustrative example is shown below in which the acid 3-10 and can be coupled to an acyl hydrazide, and the resulting intermediate can be cyclized to the oxadiazole using dehydrating agents such as phosphorous oxychloride. The cyclized product can be deprotected in the manner described in earlier schemes to afford the desired compound 6-1.

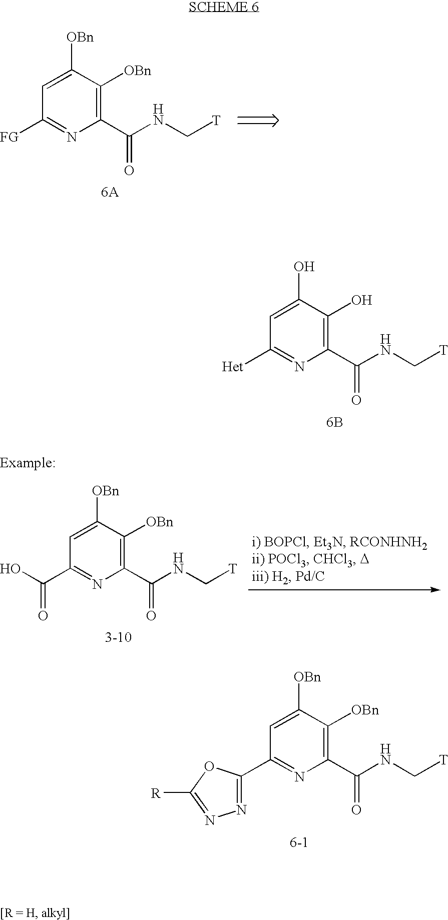

The polyfunctionalized pyridines can also be prepared as described in Scheme 7, wherein a 2-chloro-3-hydroxy pyridine can be protected as described in Theodora W. Greene and Peter G. M. Wuts, Protective Groups in Organic Synthesis, 3^{rd} Edition, Wiley-Interscience to yield 7-2 (e.g. with a benzyl group or a MOM-group). The MOM group can then be used to direct an ortho-lithiation as described in J. Org. Chem. 1994, 59, 6173-8 and the resulting lithium derivative can be quenched on solid carbon dioxide to yield the corresponding acid 7-3. This acid can be coupled with a suitable amine in the manner described in previous schemes to give 7-4. The material can be sequentially deprotected to give 7-5 and the free 3-hydroxy group on 7-5 can be used to direct iodination at C-6, as described in J. Org. Chem. 1998, 63, 7851, to provide 7-6. Palladium catalyzed cross-coupling of an organostannane as described by Jiro Tsuji, Palladium Reagents and Catalysts, Wiley p. 228 will afford an intermediate which can be deprotected using acid to yield 7-7. Alternatively, the benzyl group can be removed by hydrogenolysis to give 7-8.

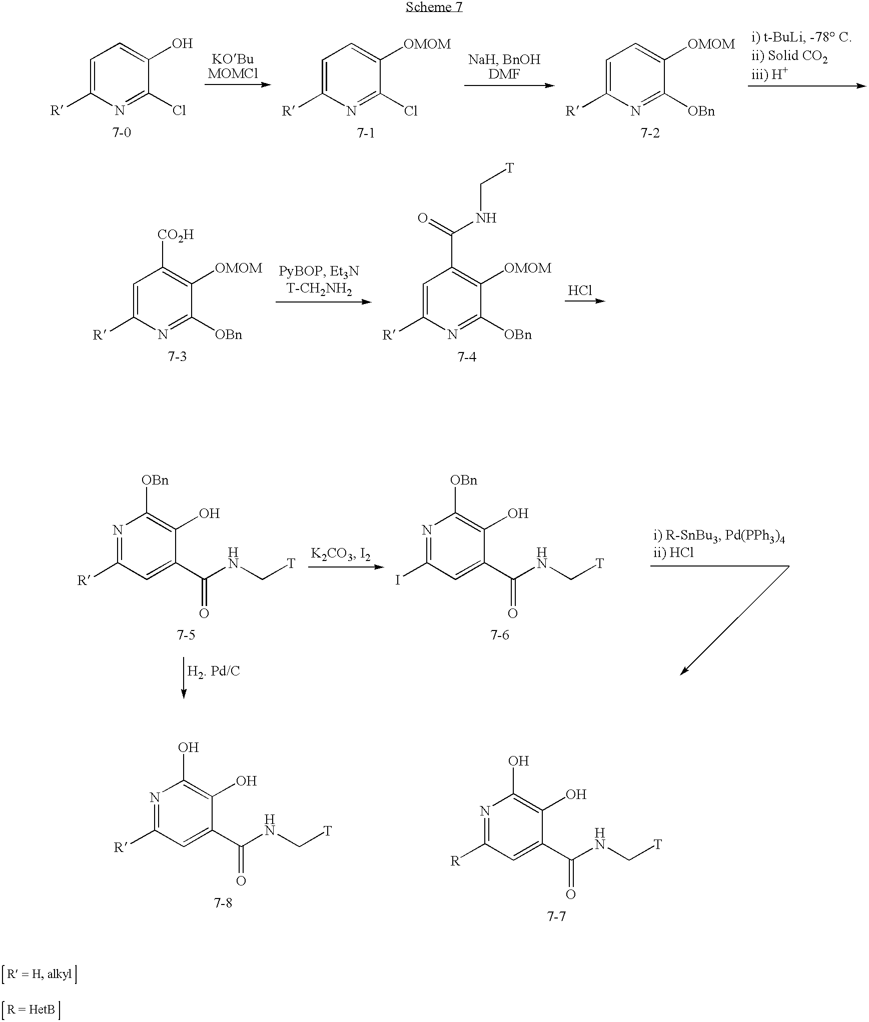

The chemistry illustrated in Scheme 8 shows how a ketone at the C-6 position of the pyridine can be reduced to the corresponding alcohol 8-1 (such as with NaBH₄) and this 5 alcohol can be then converted into a leaving group (for instance, a mesylate 8-2, a chloride or bromide see Richard Larock, Comprehensive Organic Transformations, VCH Publishers Inc, 1989). The leaving group can then be displaced using a primary or secondary amine to form compound 8-3. Deprotection as described in previous schemes provides 8-4.

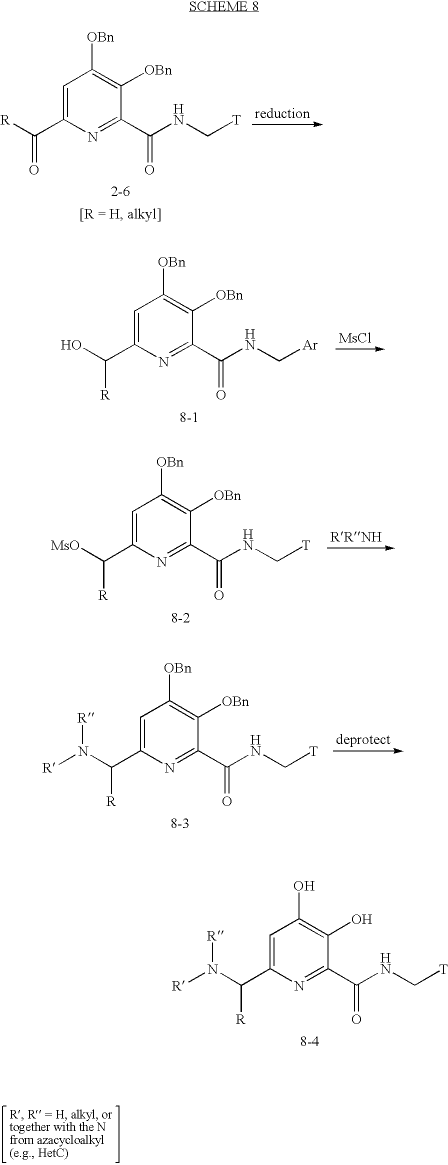

N-substituted pyridones can be prepared as depicted in Scheme 9. wherein compound 7-4 can be selectively deprotected by hydrogenation to give 9-1, which can then be N-alkylated using a suitable electrophile (e.g., an organic halide, mesylate, or tosylate) in the presence of a base (e.g., K₂CO₃) and then deprotected as described in previous schemes to afford compound 9-2.

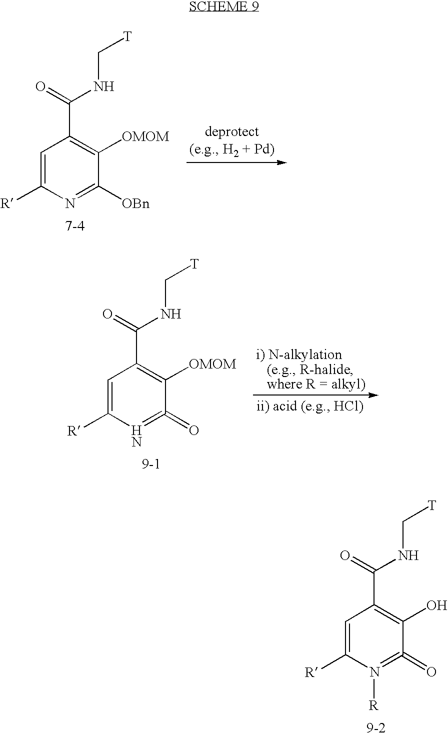

Scheme 10 depicts an alternative method to introduce a group at the C-6 position of the pyridine. Iodide 7-5 can be protected (e.g., with a benzyl group as shown), and then subjected to palladium catalyzed cross-coupling with a stannylated alkyl enol ether (see Chemistry Lett. 1989, 1959-62) to give an intermediate enol ether, which can be hydrolyzed with acid to give the corresponding ketone 10-2. This ketone can then be transformed into an amine 10-4 using the same methodology as described in Scheme 8. The amine can then either be deprotected (e.g., hydrogenated) to give 10-6, or can be reacted with a suitable capping group (Cap-Cl), such as an acyl chloride, a sulfonyl chloride, or a carbamyl chloride. These reactions are conducted in the presence of a base (e.g., triethylamine) to scavenge the HCl by-product. Deprotection will afford 10-6.

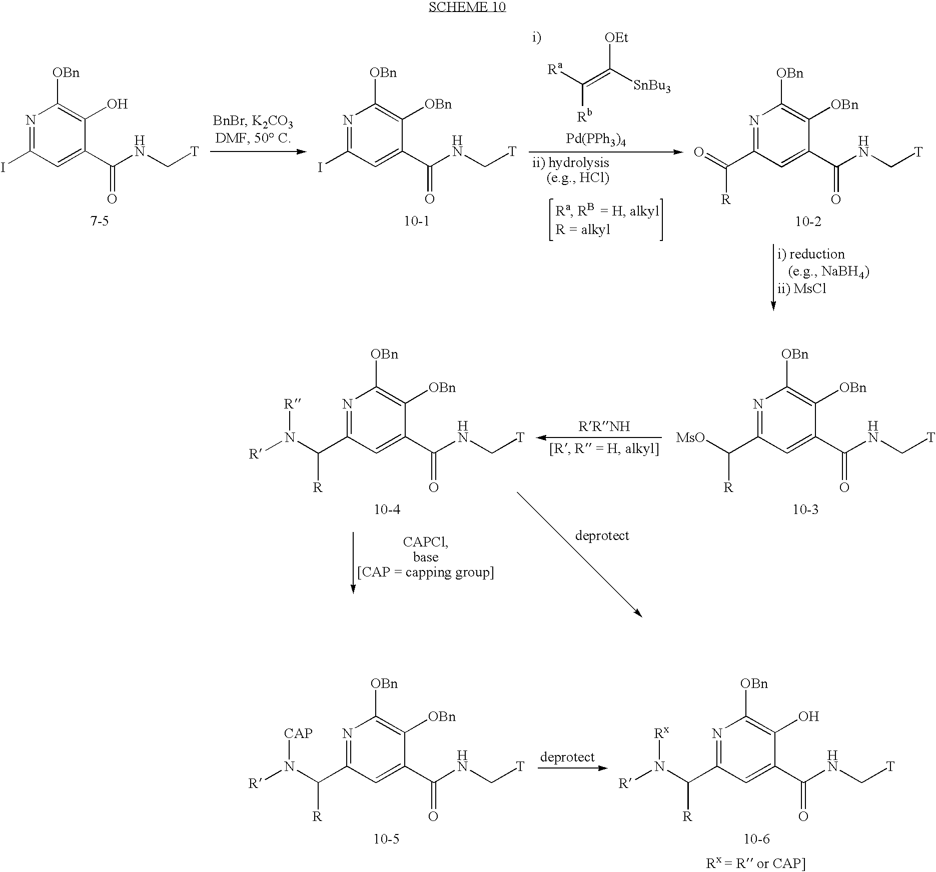

Scheme 11 presents a method of introducing heteroaryl groups at C-6 in the 5 pyridine ring, wherein intermediate 10-1 is used for Suzuki palladium catalyzed cross-coupling with organoboranes (using a Pd catalyst such as Pd/P(t-Bu)₃ and a base such as cesium carbonate at about 120° C. in a microwave) to yield compounds of the type 11-1 (see Buchwald et al., Organic Letters 2000, 2: 1729). These can be deprotected with for instance HBr in AcOH to yield compounds of the type 11-2.

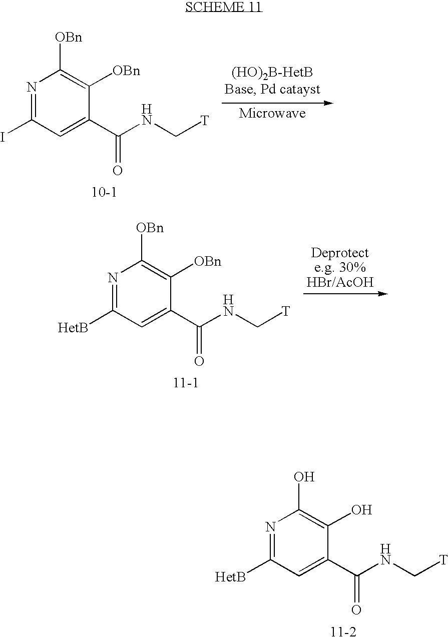

Scheme 12 depicts the reaction of the iodinated intermediate 10-1 with trifluoroiodomethane and copper, in a similar manner to that described by Humber, L. et al. J. Med. Chem. 1984, 27, 255, under microwave conditions to afford the trifluoromethyl product 12-1.

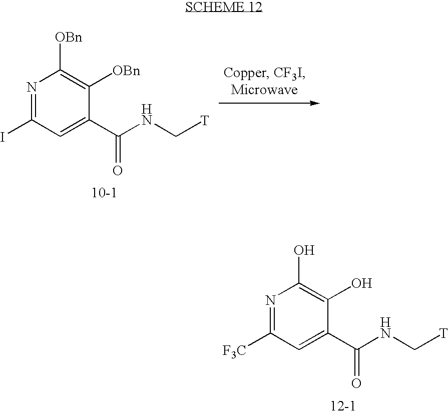

Scheme 13 depicts the conversion of the iodide 10-1 to the corresponding acid 13-1 by carbonylation with carbon monoxide in the presence of a palladium catalyst (for instance, see Jiro Tsuji, Palladium Reagents and Catalysts, Wiley, p. 188). Acid 13-1 can then be coupled to an amine to afford amide 13-2 which can be deprotected (for example using hydrogenation or HBr in HOAc) to give compounds of the type 13-3. In turn, compounds of the type 13-3 can be double alkylated with a suitable electrophile (e.g. alkyl iodide) and a base, such as cesium carbonate, to provide compounds such as 13-4 after removal of the O-alkyl group with reagents such as BBr₃.

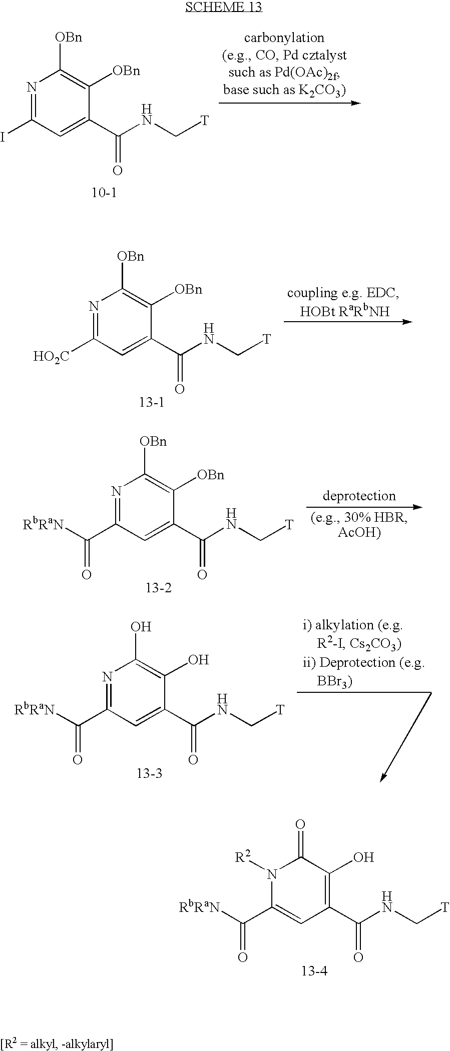

An approach to the preparation of N-benzylated compounds is depicted in Scheme 14, wherein 2,3-dihydroxypyridine is doubly alkylated to give 14-2. The O-benzyl group can then be selectively removed, for instance by hydrogenation. The 3-hydroxy group can then be used to introduce a carboxylate group at C-4 using the Kolbe-Schmitt reaction (A. S. Lindsey, H. Jeskey, Chem. Rev. 1957 (57) 583-620, K. Raymond et. al. J. Am. Chem. Soc. 1995, 117, 7245-7246, K. Raymond, J. Xu. U.S. Pat. No. 5,624,901). Conversion of the acid with methanol and thionyl chloride as described by M. Brenner and W. Huber in Helv. Chem. Acta 1953, 1109 will give the methyl ester 14-4. Reaction with a neat substituted benzylamines will afford compounds of the type 14-5.

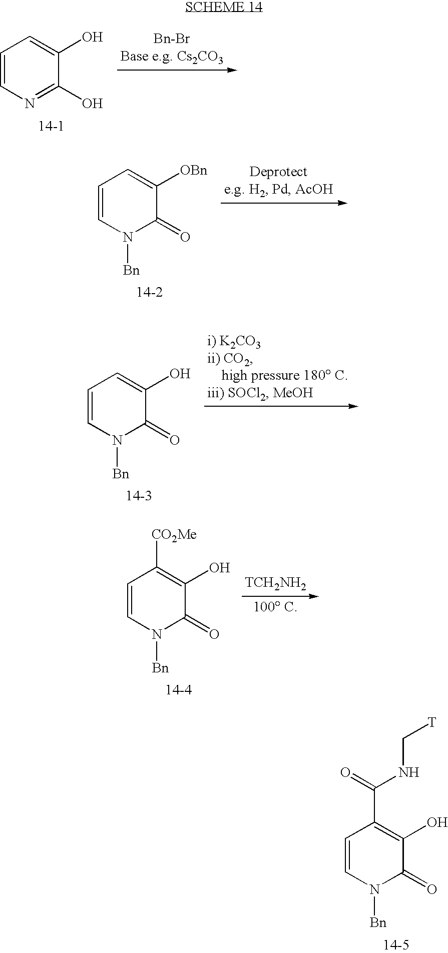

The following examples serve only to illustrate the invention and its practice. The examples are not to be construed as limitations on the scope or spirit of the invention.

### EXAMPLE 1

### 6-Acetyl-N-(4-fluorobenzyl)-3,4-dihydroxypyridine-2-carboxamide

### Step 1: [3,4-bis(Benzyloxy)-1-oxidopyridin-2-yl]methanol (A1)

mCPBA (2.0 equivalents) was added portionwise to a stirred solution of [3,4-bis(benzyloxy)pyridin-2-yl]methanol (Tetrahedron 2001, 57, 3479) (1 equivalent) in DCM at 0° C. and the mixture was stirred for 1 hour at 0° C. The cooling bath was removed and the reaction was stirred at room temperature for a further 2 hours. The reaction mixture was diluted with DCM and washed with saturated NaHCO₃ solution and then brine. The organics were concentrated under reduced pressure and purified by column chromatography on silica eluting with 4% MeOH/DCM to yield the desired pyridine-N-oxide A1.

¹H NMR (300 MHz, CDCl₃) δ 7.98 (1H, d, J=8 Hz), 7.55-7.40 (5H, m), 7.38-7.23 (5H, m), 6.83 (1H, d, J=8 Hz), 5.21 (2H, s), 5.12 (2H, s), 4.78 (2H, s).

### Step 2: 4,5-bis(Benzyloxy)-6-(hydroxymethyl)pyridine-2-carbonitrile (A2)

A solution of the pyridine-N-oxide A1 (1 equivalent) in DCM was treated with TMSCN (1.5 equivalents), and then after 5 minutes Et₂NCOCl (1.5 equivalents) was added. The resulting mixture was then stirred for a further 18 hours at room temperature after which time more TMSCN (1 equivalent) and then Et₂NCOCl (0.5 equivalent) were added. The reaction was left for a further 2 hours and then concentrated under reduced pressure. The crude residue was taken up in THF and 1N HCl added. The resulting mixture was stirred for 10 minutes and was then neutralized with 2 N NaOH solution. The product was extracted with DCM and the DCM extracts were dried (Na₂SO₄) and then concentrated under reduced pressure to yield the desired nitrile A2.

¹H NMR (300 MHz, CDCl₃) δ 7.45-7.25 (12H, m), 5.22 (2H, s), 5.16 (2H, s), 4.67 (2H, s). MS (ES) C₂₁H₁₈N₂O₃ requires: 346, found: 347 (M+H⁺).

### Step 3: 1-[4,5-bis(Benzyloxy)-6-(hydroxymethyl)pyridin-2-yl]ethanone (A3)

A solution of MeMgBr in Et₂O (5 equivalents) was added dropwise over 10 min to a stirred solution of the nitrile A2 (1 equivalent) in THF at room temperature under N₂. The reaction was stirred for 10 minutes and was then quenched cautiously with 1M HCl solution. After stirring for 10 minutes the mixture was neutralized with 2 N NaOH solution and the product was then extracted with EtOAc. The combined organics extracts were washed with brine, dried (Na₂SO₄) and concentrated under reduced pressure. The resulting ketone A3 was used without further purification.

¹H NMR (300 MHz, CDCl₃) δ 7.78 (1H, s), 7.50-7.25 (10H, m), 5.28 (2H, s), 5.18 (2H, s), 4.67 (2H, s), 2.71 (3H, s). MS (ES) C₂₂H₂₁NO4 requires: 363, found: 364 (M+H⁺).

### Step 4: 6-Acetyl-3,4-bis(benzyloxy)pyridine-2-carbaldehyde (A4)

Anhydrous DMSO (2.4 equivalents) was added dropwise over 10 min to a stirred solution of oxalyl chloride (1.2 equivalents) in dry DCM at −78° C. under N₂. The resulting mixture was then stirred at this temperature for 5 min and a solution of the above alcohol A3 (1 equivalent) in DCM was added dropwise over 20 minutes. After stirring for a further 25 min at −78° C., Et₃N (5.0 equivalents) was added dropwise over 5 minutes, the mixture was then stirred for 10 minutes and after the cooling bath was removed and the reaction was warmed to room temperature and stirred for an hour. After diluting with DCM, the mixture was washed with H₂O and then brine, dried (Na₂SO₄) and concentrated under reduced pressure. The residue was purified by column chromatography on silica eluting with 25-40% EtOAc/petroleum ether to yield the desired ketoaldehyde A4.

¹H NMR (400 MHz, CDCl₃) δ 10.25 (1H, s), 7.88 (1H, s), 7.50-7.25 (10H, m), 5.38 (4H, s), 2.81 (3H, s). MS (ES) C₂₂H₁₉NO₄ requires: 361, found: 362 (M+H⁺).

### Step 5: 6-Acetyl-3,4-bis(benzyloxy)pyridine-2-carboxylic acid (A5)

Sulfamic acid (1.4 equivalents) and then sodium chlorite (1.1 equivalents) were added sequentially to a stirred solution of the aldehyde A4 (1 equivalent) in acetone and H₂O. The resulting mixture was stirred at room temperature for 45 min and then the acetone was removed under reduced pressure. The organics were extracted with DCM, and then the DCM extracts were washed with brine, at this stage some EtOAc was added to aid solubility. The extracts were dried (Na₂SO₄) and concentrated under reduced pressure to yield the desired acid A5.

¹H NMR (400 MHz, d₆-DMSO) δ 7.81 (1H, s), 7.53 (2H, d, J=7 Hz), 7.48-7.25 (8H, m), 5.41 (2H, s), 5.14 (2H, s), 2.59 (3H, s). MS (ES) C₂₂H₁₉NO₅ requires: 377, found: 378 (M+H⁺).

### Step 6: 6-Acetyl-N-[(4-fluorophenyl)methyl]-3,4-bis-(benzyloxy)-2-pyridinecarboxamide (A6)

PyBOP (1.2 equivalents) was added to a stirred solution of the acid A5 (1 equivalent), 4-fluorobenzylamine (1.2 equivalents) and Et₃N (2.5 equivalents) in DCM and the mixture was stirred at room temperature overnight. The reaction was diluted with DCM and washed sequentially with 0.5 N HCl solution, saturated NaHCO₃ solution and brine and then dried (Na₂SO₄). The resulting solution was concentrated under reduced pressure and then purified by column chromatography on silica eluting with 35-60% EtOAc/petroleum ether to yield the desired amide A6.

¹H NMR (400 MHz, CDCl₃) δ 7.82 (1H, s), 7.77 (1H, t, J=6 Hz), 7.50-7.25 (12H, m), 7.02 (2H, t, J=8 Hz), 5.37 (2H, s), 5.31 (2H, s), 4.63 (2H, d, J=6 Hz), 2.81 (3H, s). MS (ES) C₂₉H₂₅N₂O₄F requires: 484, found: 485 (M+H⁺).

### Step 7: 6-Acetyl-N-(4-fluorobenzyl)-3,4-dihydroxypyridine-2-carboxamide (A7)

10% Pd on carbon was added to a stirred solution of the amide A6 (1 equivalent) in MeOH containing 1 M HCl solution (1 equivalent) and then after degassing the reaction vessel an H₂ atmosphere was introduced and the reaction was stirred for 2 hours. The catalyst was filtered off through celite and the filter pad washed well with MeOH. The organics were concentrated under reduced pressure and the residue was purified by reverse phase HPLC to yield the desired dihydroxypyridine A7.

¹H NMR (300 MHz, d₆-DMSO) δ 13.03 (1H, br. s), 11.12 (1H, br. s), 9.65 (1H, t, J=6 Hz), 7.50 (1H, s), 7.42 (2H, dd, J=8.8, 5.7 Hz), 7.18 (2H, t, J=8.8 Hz), 4.57 (2H, d, J=6 Hz), 2.68 (3H, s). MS (ES) C₁₅H₁₃N₂O₄F requires: 304, found: 305 (M+H⁺).

### EXAMPLE 2

### 6-{[(4-Fluorobenzyl)amino]carbonyl}-4,5-dihydroxypyridine-2-carboxylic acid

### Step 1: 3-Hydroxy-2-(hydroxymethyl)-6-[(tetrahydro-2H-pyran-2-yloxy)methyl]-4H-pyran-4-one (B1)

5-Hydroxy-2-[(tetrahydro-2H-pyran-2-yloxy)methyl]-4H-pyran-4-one (1 equivalent) (J. Med. Chem. 1988, 31, 1052) was added to a stirred solution of NaOH (1.1 equivalents) in H₂O, after 5 min when the compound had dissolved an aqueous solution of formaldehyde (30%, 1.12 equivalents) was added dropwise over 5 min. The resulting reaction mixture was stirred overnight and then neutralized with 6 N HCl. The desired material was extracted with DCM and the DCM extracts were then dried (Na₂SO₄) and concentrated under reduced pressure to yield the desired alcohol B1.

¹H NMR (300 MHz, CDCl₃) δ 6.53 (1H, s), 4.74-4.65 (1H, m), 4.68 (2H, s), 4.55 (1H, d, J=14.6 Hz), 4.39 (1H, d, J=14.6 Hz), 3.89-3.77 (1, m), 3.60-3.48 (1H, m), 1.95-1.45 (6H, m). MS (ES) C₁₂H₁₆O₆ requires: 256, found: 257 (M+H⁺).

### Step 2: 3-(Benzyloxy)-2-(hydroxymethyl)-6-[(tetrahydro-2H-pyran-2-yloxy)methyl]-4H-pyran-4-one (B2)

A mixture of the pyran B1 (1 equivalent), benzyl chloride (2 equivalents) and K₂CO₃ (2 equivalents) in DMF was heated at 130° C. for 1 hour and then was cooled to room temperature. The mixture was diluted with H₂O and then extracted with EtOAc. The EtOAc extracts were washed well with H₂O and brine, and then dried (Na₂SO₄) and concentrated under reduced pressure. The crude residue was purified by column chromatography on silica eluting with 40% EtOAc/petroleum ether to yield the desired protected material B2.

¹H NMR (300 MHz, CDCl₃) δ 7.43-7.30 (5H, m), 6.52 (1H, s), 4.72 (2H, s), 4.74-4.68 (1H, m), 4.52 (1H, d, J=14.8 Hz), 4.37-4.25 (3H, m), 3.89-3.75 (1, m), 3.58-3.48 (1H, m), 1.95-1.50 (6H, m).

### Step 3: 3-(Benzyloxy)-4-oxo-6-[(tetrahydro-2H-pyran-2-yloxy)methyl]-4H-pyran-2-carbaldehyde (B3)

Pyridine sulfur trioxide complex (5 equivalents) was added to a stirred solution of the alcohol B2 (1 equivalent) in CHCl₃, dry DMSO and Et₃N (6 equivalent) at 0° C. under N₂. The resulting mixture was warmed slowly to room temperature over 4 hours. It was then diluted with DCM and washed with H₂O and brine. After drying (Na₂SO₄), the mixture was concentrated under reduced pressure and then was purified by column chromatography on silica eluting with 40% EtOAc/petroleum ether to yield the desired aldehyde B3.

¹H NMR (300 MHz, CDCl₃) δ 9.88 (1H, s), 7.40-7.30 (5H, m), 6.62 (1H, s), 5.50 (2H, s), 4.74-4.68 (1H, m), 4.52 (1H, d, J=15.3 Hz), 4.37 (1H, d, J=15.3 Hz), 3.89-3.77 (1, m), 3.59-3.46 (1H, m), 1.87-1.50 (6H, m).

### Step 4: 3-(Benzyloxy)-4-oxo-6-[(tetrahydro-2H-pyran-2-yloxy)methyl]-4H-pyran-2-carboxylic acid (B4)

The aldehyde B3 was oxidized according to Example 1 Step 5 to yield the corresponding acid B4.

¹H NMR (300 MHz, d₆-DMSO) δ 7.53-7.30 (5H, m), 6.57 (1H, s), 5.13 (2H, s), 4.73 (1H, s), 4.47 (1H, d, J=15.5 Hz), 4.41 (1H, d, J=15.5 Hz), 3.80-3.68 (1H, m), 3.52-3.41 (1H, m), 1.79-1.40 (6H, m). MS (ES) C₁₉H₂₀O₇ requires: 360, found: 361 (M+H⁺).

### Step 5: 3-(Benzyloxy)-4-oxo-6-[(tetrahydro-2H-pyran-2-yloxy)methyl]-1,4-dihydropyridine-2-carboxylic acid (B5)

The acid B4 (1 equivalent) was dissolved in EtOH and concentrated ammonia solution was added. The mixture was stirred at room temperature for a week and was then concentrated under reduced pressure. The material B5 was used without further purification.

¹H NMR (300 MHz, CD₃OD) δ 7.85-7.70 (2H, m), 7.57-7.37 (3H, m), 6.68 (1H, s), 5.35 (2H, s), 4.95-4.72 (3H, m), 4.18-4.06 (1H, m), 3.85-3.71 (1H, m), 2.14-1.70 (6H, m). MS (ES) C₁₉H₂₁NO₆ requires: 359, found: 358 (M-H⁻).

### Step 6: Benzyl 3,4-bis(benzyloxy)-6-[(tetrahydro-2H-pyran-2-yloxy)methyl]pyridine-2-carboxylate (B6)

The crude residue from above B5 (1 equivalent) was taken up in DMF and K₂CO₃ (3 equivalents) and benzyl bromide (2.2 equivalents) were added. The reaction was stirred at room temperature for 48 hours and then more benzyl bromide (1.1 equivalents) was added and the reaction was heated at 70° C. for 3 hours. The mixture was then concentrated under reduced pressure whilst azeotroping with xylene. H₂O was added to the residue and the desired material was extracted with EtOAc. The EtOAc extracts were washed with H₂O and brine, then dried (Na₂SO₄) and concentrated under reduced pressure. The crude residue was purified by column chromatography on silica eluting with 35-40% EtOAc/petroleum ether to yield the desired protected material B6.

¹H NMR (300 MHz, CDCl₃) δ 7.47-7.27 (16H, m), 5.37 (2H, s), 5.26 (2H, s), 5.03 (2H, s), 4.86 (1H, d, J=13.9 Hz), 4.73-4.65 (1H, m), 4.59 (1H, d, J=13.9 Hz), 3.92-3.69 (1H, m), 3.59-3.44 (1H, m), 1.95-1.45 (6H, m). MS (ES) C₃₃H₃₃NO₆ requires: 539, found: 540 (M+H⁺).

### Step 7: 3,4-bis(Benzyloxy)-N-(4-fluorobenzyl)-6-[(tetrahydro-2H-pyran-2-yloxy)methyl]pyridine-2-carboxamide (B7)

A mixture of the above ester B6 (1 equivalent) and 4-fluorobenzylamine (10 equivalents) were heated at 110° C. for 90 min. After cooling to room temperature the mixture was purified by column chromatography on silica eluting with 60% EtOAc/petroleum ether to yield the desired amide B7. ¹H NMR (300 MHz, CDCl₃) δ 8.05-7.93 (1H, m), 7.47-7.17 (13H, m), 6.96 (2H, t, J=8.8 Hz), 5.21 (2H, s), 5.16 (2H, s), 4.78 (1H, d, J=13.7 Hz), 4.70-4.64 (1H, m), 4.61-4.52 (3H, m), 3.89-3.76 (1H, m), 3.56-3.43 (1H, m), 1.89-1.48 (6H, m). MS (ES) C₃₃H₃₃N₂O₅F requires: 556, found: 557 (M+H⁺).

### Step 8: 3,4-bis(Benzyloxy)-N-(4-fluorobenzyl)-6-(hydroxylmethyl)pyridine-2-carboxamide (B8)

The amide B7 (1 equivalent) was taken up in THF and treated with 1M HCl solution. The mixture was stirred at room temperature for 1 hour and was subsequently neutralized with 1 M NaOH solution. The organics were extracted with EtOAc, dried (Na₂SO₄), and concentrated under reduced pressure to yield the desired alcohol B8.

¹H NMR (300 MHz, CDCl₃) δ 7.85-7.75 (1H, m), 7.43-7.20 (12H, m), 7.07-6.93 (3H, m), 5.18 (2H, s), 5.07 (2H, s), 4.69 (2H, s), 4.56 (2H, d, J=6 Hz). MS (ES) C₂₈H₂₅N₂O₄F requires: 472, found: 473 (M+H⁺).

### Step 9: 3,4-bis(Benzyloxy)-N-(4-fluorobenzyl)-6-formylpyridine-2-carboxamide (B9)

The alcohol B8 was oxidized according to Example 2 Step 3. and the resulting residue was purified by column chromatography on silica eluting with 45-60% EtOAc/petroleum ether to yield the desired aldehyde B9.

¹H NMR (300 MHz, CDCl₃) δ 9.93 (1H, s), 7.88 (1H, br. s), 7.77 (1H, s), 7.45-7.18 (12H, m), 7.04 (2H, t, J=8.8 Hz), 5.27 (2H, s), 5.23 (2H, s), 4.60 (2H, d, J=6 Hz). MS (ES) C₂₈H₂₃N₂O₄F requires: 470, found: 471 (M+H⁺).

### Step 10: 4,5-bis(Benzyloxy)-6-{[(4-fluorobenzyl)amino]carbonyl}pyridine-2-carboxylic acid (B10)

The aldehyde B9 was oxidized according to Example 1 Step 5 to yield the desired acid B10.

¹H NMR (300 MHz, d₆-DMSO) δ 13.05 (1H, br. s), 9.21 (1H, t, J=6 Hz), 7.90 (1H, s), 7.57-7.20 (12H, m), 7.09 (2H, t, J=8.8 Hz), 5.39 (2H, s), 5.08 (2H, s), 4.47 (2H, d, J=6 Hz). MS (ES) C₂₈H₂₃N₂O₅F requires: 486, found: 487 (M+H⁺).

### Step 11: N-(4-fluorobenzyl)-6-carboxyl-3,4-dihydroxy-pyridine-2-carboxamide (B11)

10% Pd on carbon was added to a stirred solution of the acid B10 (1 equivalent) in MeOH and then after degassing the reaction vessel an H₂ atmosphere was introduced and the reaction was stirred for 1 hours. The catalyst was filtered off through celite and the filter pad washed well with MeOH. The organics were concentrated under reduced pressure and the residue was triturated with hexanes and filtered. The resulting solid was dried under vacuum yielding the desired dihydroxypyridine B11.

¹H NMR (300 MHz, d₆-DMSO) δ 12.85 (1H, br. s), 10.05-9.95 (1H, m), 7.61 (1H, s), 7.44-7.33 (2H, m), 7.07 (2H, t, J=8.8 Hz), 4.55 (2H, d, J=6 Hz). MS (ES) C₁₄H₁₁N₂O₅F requires: 306, found: 307 (M+H⁺).

### EXAMPLE 3

### Methyl 6-{[(4-fluorobenzyl)amino]-carbonyl}-4,5-dihydroxypyridine-2-carboxylate

### Step 1: Methyl 4,5-bis(benzyloxy)-6-{[(4-fluorobenzyl)amino]carbonyl}pyridine-2-carboxylate (C1)

4,5-bis(Benzyloxy)-6-{[(4-fluorobenzyl)amino]carbonyl}pyridine-2-carboxylic acid B10 (1 equivalent) (Example 2 Step 10) was taken up in MeOH and a solution of trimethylsilyl diazomethane in hexanes (3 equivalents) was added dropwise over 5 minutes. The resulting solution was stirred overnight and then was concentrated under reduced pressure. The resulting ester C1 was used without further purification.

¹H NMR (300 MHz, CDCl₃) δ 7.95-7.85 (2H, m), 7.45-7.18 (12H, m), 6.98 (2H, t, J=8.8 Hz), 5.27 (2H, s), 5.23 (2H, s), 4.63 (2H, d, J=6 Hz), 3.93 (3H, s). MS (ES) C₂₉H₂₅N₂O₅F requires: 500, found: 501 (M+H⁺).

### Step 2: Methyl 6-{[(4-fluorobenzyl)amino]-carbonyl}-4,5-dihydroxypyridine-2-carboxylate (C2)

10% Pd on carbon was added to a stirred solution of the ester C1 (1 equivalent) in MeOH and EtOAc, then after degassing the reaction vessel an H₂ atmosphere was introduced and the reaction was stirred for 1 hour. The catalyst was filtered off through celite and the filter pad washed well with MeOH. The organics were concentrated under reduced pressure and the residue was triturated with hexanes and filtered. The resulting solid was dried under vacuum, yielding the desired dihydroxypyridine C2.

¹HNMR (300 MHz, d₆-DMSO) 69.38 (1H, t, J=6 Hz), 7.61 (1H, s), 7.44-7.36 (2H, m), 7.13 (2H, t, J=8.8 Hz), 4.53 (2H, d, J=6 Hz), 3.85 (3H, s). MS (ES) C₁₅H₁₃N₂O₅F requires: 320, found: 321 (M+H⁺).

### EXAMPLE 4

### N²-(4-Fluorobenzyl)-3,4-dihydroxy-N⁶-(pyridin-3-ylmethyl)pyridine-2,6-dicarboxamide

### Step 1: 3,4-bis(Benzyloxy)-N²-(4-fluorobenzyl)-N⁶-(pyridin-3-yl methyl)pyridine-2,6-dicarboxamide (D1)

4,5-bis(Benzyloxy)-6-{[(4-fluorobenzyl)amino]carbonyl}pyridine-2-carboxylic acid B10 (1 equivalent) (Example 2 Step 10) was taken up in DCM and 3-aminomethylpyridine (1.3 equivalents), Et₃N (1.5 equivalents) and finally BOPCl (1.3 equivalents) were added. The reaction was stirred at room temperature for 3 hours and was then diluted with DCM and washed with saturated NaHCO₃ solution. The DCM layer was concentrated under reduced pressure and crude residue then was purified by column chromatography on silica eluting with 3% MeOH/DCM to yield the desired bis-amide D1. MS (ES) C₃₄H₂₉N₄O₄F requires: 576, found: 577 (M+H⁺).

### Step 2: N²-(4-Fluorobenzyl)-3,4-dihydroxy-N⁶-(pyridin-3-ylmethyl)pyridine-2,6-dicarboxamide (D2)

10% Pd on carbon was added to a stirred solution of the amide D1 (1 equivalent) in MeOH and EtOAc, then after degassing the reaction vessel an H₂ atmosphere was introduced and the reaction was stirred at room temperature for 2.5 hours. The catalyst was filtered off through celite and the filter pad washed well with MeOH. The organics were concentrated under reduced pressure and the subsequent residue was triturated with hexanes and filtered. The resulting solid was dried under vacuum, yielding the desired dihydroxypyridine D2. ¹H NMR (300 MHz, d₆-DMSO) δ 10.13-10.00 (1H, m), 9.68-9.55 (1H, m), 8.56 (1H, s), 8.45-8.38 (1H, m), 7.67 (1H, d, J=7.7 Hz), 7.59 (1H, s), 7.45-7.31 (3H, m), 7.13 (2H, t, J=8.8 Hz), 4.62-4.48 (4H, m). MS (ES) C₂₀H₁₇N₄O₄F requires: 396, found: 395 (M-H⁻).

### EXAMPLE 5

### N-(4-Fluorobenzyl)-3,4-dihydroxy-6-(5-methyl-1,3,4-oxadiazol-2-yl)pyridine-2-carboxamide

### Step 1: 3,4-bis(Benzyloxy)-N-(4-fluorobenzyl)-6-(5-methyl-1,3,4-oxadiazol-2-yl)pyridine-2-carboxamide (E1)

4,5-bis(Benzyloxy)-6-{[(4-fluorobenzyl)amino]carbonyl}pyridine-2-carboxylic acid B10 (1 equivalent) (Example 2 Step 10) was taken up in DCM and acetyl hydrazide (1.2 equivalents), Et₃N (2.0 equivalents) and finally BOPCl (1.2 equivalents) were added. The reaction mixture was stirred at room temperature for 2 hours and was then diluted with DCM and washed 0.5 N NaOH solution. The DCM layer was dried (Na₂SO₄) and concentrated under reduced pressure. MS (ES) C₃₀H₂₇N₄O₅F requires: 542, found: 543 (M+H₊). The crude residue was then taken up in CHCl₃, and POCl₃ (7 equivalents) was added. The mixture was then heated at reflux for 3 hours and then at 60° C. overnight. It was then diluted with DCM and then 0.5 N NaOH solution was added and the resulting mixture was stirred at room temperature for 30 min. The phases were separated and the aqueous layer was extracted with more DCM. The combined DCM layers were washed with brine, dried (Na₂SO₄) and concentrated under reduced pressure. The residue was purified by column chromatography on silica eluting with 50-100% EtOAc/petroleum ether to yield the desired oxadiazole E1. MS (ES) C₃₀H₂₅N₄O₄F requires: 524, found: 525 (M+H⁺).

### Step 2: N-(4-Fluorobenzyl)-3,4-dihydroxy-6-(5-methyl-1,3,4-oxadiazol-2-yl)pyridine-2-carboxamide (E2)

10% Pd on carbon was added to a stirred solution of the oxadiazole E1 (1 equivalent) in MeOH and EtOAc, then after degassing the reaction vessel an H₂ atmosphere was introduced and the reaction was stirred at room temperature for 2 hours. The catalyst was filtered off through celite and the filter pad washed well with MeOH. The organics were concentrated under reduced pressure and the residue was purified by reverse phase HPLC to yield the desired dihydroxypyridine E2.

¹H NMR (300 MHz, d₆-DMSO) δ 12.94 (1H, br. s), 11.44 (1H, br. s), 9.49, 9.40 (1H, m), 7.63 (1H, s), 7.46-7.37 (2H, m), 7.14 (2H, t, J=8.8 Hz), 4.56 (2H, d, J=6 Hz), 2.62 (3H, s). MS (ES) C₁₆H₁₃N₄O₄F requires: 344, found: 345 (M+H⁺).

### EXAMPLE 6

### N-[(4-Fluorophenyl)methyl]-2,3-dihydroxy-6-(2-thienyl)-4-pyridine carboxamide

### Step 1: 2-Chloro-3-(methoxymethoxy)pyridine (F1)

Potassium tert-butoxide (1.2 equivalents) was added to a stirred solution of 2-chloropyridin-3-ol (1 equivalent) in DMF at 0° C. under N₂ over 5 min. The mixture was stirred for 10 min and then MOMCl (1.4 equivalents) was added dropwise over 5 min. The reaction mixture was stirred overnight gradually warming to room temperature. It was then concentrated under reduced pressure while azeotroping with xylene. H₂O was added, and the organics were extracted with EtOAc. The combined EtOAc extracts were washed with 2N NaOH and brine, then dried (Na₂SO₄) and concentrated under reduced pressure. The oily residue was left to stand at room temperature overnight and a solid crystallized from the residue. The solid, the desired pyridine F1, was isolated from the residue by decanting off the undesired oil. ¹H NMR (400 MHz, CDCl₃) δ 8.05 (1H, dd, J=4.8, 1.4 Hz), 7.49 (1H, dd, J=8.1, 1.4 Hz), 7.18 (1H, dd, J=8.1, 4.8 Hz), 5.27 (2H, s), 3.54 (3H, s).

### Step 2: 2-(Benzyloxy)-3-(methoxymethoxy)pyridine (F2)

NaH was added portionwise over 30 min to a stirred solution of benzyl alcohol (4 equivalents) in dry DMF at room temperature under N₂. Upon complete addition the mixture was stirred for a further hour and then a solution of the above chloride F1 (1 equivalent) in DMF was added. The mixture was heated at 90° C. for 5 hours and then cooled to room temperature. The solvent was removed under reduced pressure whilst azeotroping with xylene. The residue was taken up in Et₂O and washed with saturated NH₄Cl solution and then brine. The Et₂O layer was dried (Na₂SO₄), and concentrated under reduced pressure. The crude residue was purified by column chromatography on silica eluting with 6-10% EtOAc/petroleum ether to yield the desired pyridine F2.

¹H NMR (400 MHz, CDCl₃) δ 7.84 (1H, dd, J=5.0, 1.5 Hz), 7.48 (2H, d, J=7.2 Hz), 7.40-7.26 (4H, m), 6.83 (1H, dd, J=7.9, 5.0 Hz), 5.51 (2H, s), 5.26 (2H, s), 3.51 (3H, s).

### Step 3: 2-(Benzyloxy)-3-(methoxymethoxy)isonicotinic acid (F3)

A solution of tert-BuLi in Et₂O (1.4 equivalent) was added dropwise over 5 minutes to a solution of the pyridine F2 (1 equivalent) in dry Et₂O at −78° C. under N₂. A precipitate formed immediately and the resulting suspension was then stirred for a further 20 min. Solid CO₂ was then added and the cooling bath removed so the reaction could warm to room temperature. Upon reaching room temperature the reaction mixture was quenched with 1 M HCl solution and was then extracted with EtOAc. The combined organic extracts were dried (Na₂SO₄) and concentrated under reduced pressure to yield crude acid F3 which was used without further purification.

¹H NMR (300 MHz, CDCl₃) δ 8.05 (1H, d, J=5.1 Hz), 7.53-7.30 (6H, m), 5.51 (2H, s), 5.38 (2H, s), 3.51 (3H, s). MS(ES) C₁₅H₁₅NO₅ requires: 289, found: 288 (M-H⁻).

### Step 4: 2-(Benzyloxy)-N-(4-fluorobenzyl)-3-(methoxymethoxy)isonicotinamide (F4)

The above crude acid F3 was coupled with 4-fluorobenzylamine as described in Example 1 Step 6 and the crude residue was purified by column chromatography on silica eluting with 35% EtOAc/petroleum ether to yield the desired amide F4. ¹H NMR (400 MHz, d₆-DMSO) δ 8.93 (1H, t, J=6.0 Hz), 7.96 (1H, d, J=5.1 Hz), 7.46 (2H, d, J=7.1 Hz), 7.40-7.25 (5H, m), 7.16 (2H, t, J=8.9 Hz), 7.05 (1H, d, J=5.1 Hz), 5.39 (2H, s), 5.09 (2H, s), 4.43 (2H, d, J=6.0 Hz), 3.26 (3H, s). MS(ES) C₂₂H₂₁FN₂O₄ requires: 396, found: 397 (M+H⁺).

### Step 5: 2-(Benzyloxy)-N-(4-fluorobenzyl)-3-hydroxy-6-iodoisonicotinamide (F5)

A mixture of the amide F4 (1 equivalent) in THF and 1M HCl (5 equivalents) was stirred at 60° C. for 2.5 hours. The mixture was cooled to room temperature and quenched with 2N NaOH solution (5 equivalents). MS (ES) C₂₀H₁₇FN₂O₃ requires: 352, found: 353 (M+H⁺). K₂CO₃ (2 equivalents) was added to this solution, followed by iodine (2 equivalents) and the mixture was stirred at room temperature for 30 min. The reaction was neutralized with 1M HCl solution and extracted with DCM. The combined DCM extracts were washed with saturated sodium thiosulfate solution and were then dried (Na₂SO₄). After concentration under reduced pressure, the residue was purified by column chromatography on silica eluting with 50% EtOAc/petroleum ether to yield the desired iodide F5.

¹H NMR (400 MHz, CDCl₃) δ 10.83 (1H, s), 7.47 (2H, d, J=7.2 Hz), 7.37-7.24 (6H, m), 7.04 (2H, t, J=8.6 Hz), 6.92-6.83 (1H, m), 5.43 (2H, s), 4.56 (2H, d, J=5.7 Hz). MS(ES) C₂₀H₁₆FIN₂O₃ requires: 477, found: 476 (M-H⁻).

### Step 6: N-[(4-Fluorophenyl)methyl]-2,3-dihydroxy-6-(2-thienyl)-4-pyridinecarboxamide (F6)

A mixture of the iodide F5 (1 equivalent) and 2-thienyltributylstannane (3 equivalents) and Pd(PPh₃)₄ (10 mol %) in DMF was heated at 90° C. overnight under N₂. The solvent was removed under reduced pressure whilst azeotroping with xylene and the residue was purified by column chromatography on silica eluting with 25% EtOAc/petroleum ether to yield the desired pyridine. MS (ES) C₂₄H₁₉FN₂O₃S requires: 434, found: 435 (M+H⁺). This resulting material was taken up in THF and treated with 6 N HCl; this mixture was heated at 60° C. for 12 hours and was subsequently concentrated under reduced pressure. The residue was purified by reverse phase HPLC to yield the desired thiophene F6.

¹H NMR (400 MHz, d₆-DMSO) δ 9.02 (1H, br. s), 7.78-7.52 (2H, m), 7.38 (2H, t, J=7.8 Hz), 7.27-7.12 (3H, m), 6.76 (1H, br. s), 4.53 (2H, d, J=6 Hz). MS(ES) C₁₇H₁₃FN₂O₃S requires: 344, found: 345 (M+H⁺).

### EXAMPLE 7

### 6-[1-(Dimethylamino)ethyl]-N-(4-fluorobenzyl)-3,4-dihydroxy-pyridine-2-carboxamide, TFA salt

### Step 1: 3,4-bis(Benzyloxy)-N-(4-fluorobenzyl)-6-(1-hydroxyethyl)pyridine-2-carboxamide (G1)

Sodium borohydride (2 equivalents) was added to a stirred solution of the 6-acetyl-N-[(4-fluorophenyl)methyl]-3,4-bis-(benzyloxy)-2-pyridinecarboxamide A6 in EtOH and the resulting mixture was stirred at room temperature for 30 min. The solvent was removed under reduced pressure and then saturated aqueous NH₄Cl solution was added and the organics were extracted with DCM. The organic extracts were concentrated under reduced pressure and were purified by column chromatography on silica eluting with 5-7% MeOH/DCM to yield the desired alcohol G1. ¹H NMR (400 MHz, CDCl₃) δ 7.88-7.72 (1H, m), 7.55-7.20 (12H, m), 7.13 (1H, s), 7.06 (2H, t, J=8.6 Hz), 5.25 (2H, s), 5.15 (2H, s), 4.89 (1H, q, J=7.1 Hz), 4.62 (2H, d, J=6.0 Hz), 1.53 (3H, d, J=7.1 Hz). MS(ES) C₂₉H₂₇N₂O₄F requires: 486, found: 487 (M+H⁺).

### Step 2: 1-(4,5-bis(Benzyloxy)-6-{[(4-fluorobenzyl)amino]carbonyl}pyridin-2-yl)ethyl methanesulfonate (G2)

To an ice cold solution of the alcohol G1 (1 equivalent) and Et₃N (1.5 equivalents) in DCM was added MsCl (1.5 equivalents) dropwise over 3 min. The resulting solution was warmed to room temperature and stirred at room temperature for 1 hour and was then diluted with DCM, washed with saturated NaHCO₃ solution and brine. The organics were dried (Na₂SO₄) and the concentrated under reduced pressure to yield the crude mesylate G2.

### Step 3: 3,4-bis(Benzyloxy)-6-[1-(dimethylamino)ethyl]-N-(4-fluorobenzyl)pyridine-2-carboxamide (G3)

A mixture of the crude mesylate G2 (1 equivalent) and a 2 M solution of Me₂NH in THF (25 equivalents) were heated in a sealed tube at 75° C. for 14 hours. The mixture was diluted with DCM and washed with saturated NaHCO₃ solution, H₂O and brine. The organics were dried (Na₂SO₄) and the concentrated under reduced pressure to yield the desired amine G3. MS(ES) C₃₁H₃₂FN₃O₃ requires: 513, found: 514 (M+H⁺).

### Step 4: 6-[1-(Dimethylamino)ethyl]-N-(4-fluorobenzyl)-3,4-dihydroxypyridine-2-carboxamide, TFA salt (G4)

10% Pd on carbon was added to a stirred solution of the amide G3 (1 equivalent) in EtOH and 1M HCl (2 equivalents), then after degassing the reaction vessel an H₂ atmosphere was introduced and the reaction was stirred at room temperature for 2 hours. The catalyst was filtered off through celite and the filter pad washed well with EtOH. The organics were concentrated under reduced pressure and the subsequent residue was purified by reverse phase HPLC to yield the desired amine G4 as a TFA salt. ¹H NMR (400 MHz, d₆-DMSO) δ 12.45 (1H, s), 11.18 (1H, s), 9.67 (1H, t, J=6.0 Hz), 9.44 (1H, br. s), 7.43-7.35 (2H, m), 7.19 (2H, t, J=8.8 Hz), 7.07 (1H, s), 4.61 (2H, d, J=6.0 Hz), 4.53-4.40 (1H, m), 2.82 (3H, s), 2.75 (3H, s), 1.54 (3H, d, J=7.2 Hz). MS(ES) C₁₇H₂₀FN₃O₃ requires: 333, found: 334 (M+H⁺).

### EXAMPLE 8

### N-(4-Fluorobenzyl)-3-hydroxy-1-methyl-2-oxo-1,2-dihydropyridine-4-carboxamide

### Step 1: N-(4-fluorobenzyl)-3-(methoxymethoxy)-2-oxo-1,2-dihydropyridine-4-carboxamide (H1)

10% Pd on carbon was added to a stirred solution of 2-(benzyloxy)-N-(4-fluorobenzyl)-3-(methoxymethoxy)isonicotinamide F4 (1 equivalent) in MeOH, then after degassing the reaction vessel an H₂ atmosphere was introduced and the reaction was stirred at room temperature for 105 min. The catalyst was filtered off through celite and the filter pad washed well with MeOH. The organics were concentrated under reduced pressure to yield the 2-pyridone H1. MS(ES) C₁₅H₁₅FN₂O₄ requires: 306, found: 305 (M-H⁻).

### Step 2: N-(4-fluorobenzyl)-3-hydroxy-1-methyl-2-oxo-1,2-dihydropyridine-4-carboxamide (H3)

MeI (7 equivalents) was added to a stirred mixture of the pyridone H1 (1 equivalent), K₂CO₃ (3 equivalents) in MeOH and the mixture was stirred at room temperature for 12 hours. The reaction was neutralized with 1 M HCl solution and then the MeOH was removed under reduced pressure. The organics were extracted with DCM and then these DCM extracts were concentrated under reduced pressure. The residue was purified by column chromatography on silica eluting with 4% MeOH/DCM to yield the desired N-methylated pyridone H₂. MS (ES) C₁₆H₁₇FN₂O₄ requires: 320, found: 343 (M+Na⁺). A mixture of the N-methylpyridone H₂ (1 equivalent) in THF was treated with 1 M HCl; this mixture was heated at reflux for 5 hours and was subsequently cooled to room temperature and neutralized with 2 N NaOH. The organics were extracted with DCM and these extracts were concentrated under reduced pressure. The residue was purified by reverse phase HPLC to yield the desired pyridone H₃.

¹H NMR (400 MHz, d₆-DMSO) δ 11.52 (1H, br. s), 8.84 (1H, t, J=6.1 Hz), 7.36 (2H, dd, J=8.9, 5.7 Hz), 7.20 (1H, d, J=7.3 Hz), 7.15 (2H, t, J=8.9 Hz), 6.52 (1H, d, J=7.3 Hz), 4.48 (2H, d, J=6.1 Hz), 3.48 (3H, s). MS(ES) C₁₄H₁₃FN₂O₃ requires: 276, found: 277 (M+H⁺).

### EXAMPLE 9

### 6-{1-[Acetyl(methyl)amino]ethyl}-N-(4-fluorobenzyl)-2,3-dihydroxyisonicotinamide

### Step 1: 2,3-bis(Benzyloxy)-N-(4-fluorobenzyl)-6-iodoisonicotinamide (I1)

2-(Benzyloxy)-N-(4-fluorobenzyl)-3-hydroxy-6-iodoisonicotinamide F5 (1 equivalent) was taken up in DMF and K₂CO₃ (2 equivalents) and benzyl bromide (1.2 equivalents) were added. The reaction was heated at 50° C. for 1.5 hours. The mixture was then neutralized with 1 M HCl and then concentrated under reduced pressure whilst azeotroping with xylene. The resulting residue was dissolved in DCM and then concentrated under reduced pressure whilst dry loading onto silica. The crude residue was purified by column chromatography on silica eluting with 15% EtOAc/petroleum ether to yield the desired protected material 11.

¹H NMR (300 MHz, CDCl₃) δ 8.08-8.00 (1H, m), 7.96 (1H, s), 7.55-7.47 (2H, m), 7.44-7.21 (6H, m), 7.17-7.06 (4H, m), 6.96 (2H, t, J=8.8 Hz), 5.47 (2H, s), 5.04 (2H, s), 4.38 (2H, d, J=6.0 Hz). MS (ES) C₂₇H₂₂FIN₂O₃ requires: 567, found: 568 (M+H⁺).

### Step 2: 6-Acetyl-2,3-bis(benzyloxy)-N-(4-fluorobenzyl)isonicotinamide (I2)

The iodide I1 (1 equivalent) was cross-coupled with 2-ethoxyvinyltributyl stannane in a manner similar to that described in Example 6 Step 6. The crude residue, obtained after azeotroping with xylene, was taken up in THF and treated with 1 M HCl at room temperature for 40 min. The solution was neutralized with 2 N NaOH solution and extracted with EtOAc. The combined organic extracts were washed with brine, dried (Na₂SO₄) and concentrated under reduced pressure to yield crude methyl ketone I2. MS (ES) C₂₉H₂₅FN₂O₄ requires: 484, found: 485 (M+H⁺).

### Step 3: 1-(5,6-bis(Benzyloxy)-4-{[(4-fluorobenzyl)amino]carbonyl}pyridin-2-yl)ethyl methanesulfonate (I3)

The crude methyl ketone I2 was transformed to the mesylate I3 according to Example 7 Steps 1 and 2, (the intermediate alcohol was purified by column chromatography on silica eluting with 40-50% EtOAc/petroleum ether), to yield the mesylate I3.

¹H NMR (400 MHz, CDCl₃) δ 8.18-8.08 (1H, m), 7.69 (1H, s), 7.55-7.47 (2H, d, J=8.3 Hz), 7.43-7.25 (6H, m), 7.18-7.09 (4H, m), 6.97 (2H, t, J=8.8 Hz), 5.72 (1H, q, J=6.6 Hz), 5.52 (2H, s), 5.12 (2H, s), 4.42 (2H, d, J=2.9 Hz), 2.86 (3H, s), 1.73 (3H, d, J=6.6 Hz).

### Step 4: 2,3-bis(Benzyloxy)-N-(4-fluorobenzyl)-6-[1-(methylamino)ethyl]isonicotinamide (I4)

The mesylate I3 (1 equivalent) was reacted with MeNH₃⁺ Cl⁻ (10 equivalents) and Et₃N (10 equivalents) in DMSO in a sealed tube at 60° C. for 36 hours. DCM was added and the mixture was washed with saturated aqueous NaHCO₃ solution and H₂O and dried (Na₂SO₄). The solvent was removed under reduced pressure to give the crude amine I4. MS(ES) C₃₀H₃₀FN₃O₃ requires: 499, found: 500 (M+H⁺).

### Step 5: 6-{1-[Acetyl(methyl)amino]ethyl}-2,3-bis(benzyloxy)-N-(4-fluorobenzyl)isonicotinamide (I5)

The crude amine I4 (1 equivalent) was taken up in DCM and reacted with AcCl (4 equivalents) and Et₃N (4 equivalents) at room temperature for 2 hours. DCM was added and the mixture was washed with saturated aqueous NaHCO₃ solution, and brine. The solvent was removed under reduced pressure and the crude residue was purified by column chromatography on silica eluting with 25-100% EtOAc/petroleum ether to yield the desired acetamide 15. MS(ES) C₃₂H₃₂FN₃O₄ requires: 541, found: 542 (M+H³⁰).

### Step 6: 6-{1-[Acetyl(methyl)amino]ethyl}-N-(4-fluorobenzyl)-2,3-dihydroxyisonicotinamide (I6)

The acetamide I5 was deprotected according to Example 7 Step 4 to yield after reverse phase HPLC purification the desired pyridine I6. ¹H NMR (300 MHz, d₆-DMSO) Major rotamer: δ 11.95 (1H, br. s), 11.45 (1H, br. s), 9.05-8.95 (1H, m), 7.45-7.33 (2H, m), 7.14 (2H, t, J=8.8 Hz), 6.45 (1H, s), 5.43 (1H, q, J=7.0 Hz), 4.48 (2H, d, J=6.0 Hz), 2.77 (3H, s), 2.05 (3H, s), 1.30 (3H, d, J=7.0 Hz). MS (ES) C₁₈H₂₀FN₃O₄ requires: 361, found: 362 (M+H⁺).

### EXAMPLE 10

### 1-Benzyl-N-(2,3-dimethoxybenzyl)-3-hydroxy-2-oxo-1,2-dihydropyridine-4-carboxamide

### Step 1:1-Benzyl-3-(benzyloxy)pyridine-2(1H)-one (JI)

2,3-Dihydroxypyridine (1 equivalent) was dissolved in DMF and cesium carbonate (3 equivalents) was added. Benzyl bromide (2.5 equivalents) was added and the reaction stirred at room temperature overnight. The crude reaction was filtered and the solvent removed under reduced pressure. The residue was partitioned between Et₂O and water. The Et₂O layer was washed with water several times, dried (Mg₂SO₄) and evaporated to give the crude product as a brown solid J1 which was used in the next reaction.

¹H NMR (CDCl₃, 400 MHz,) δ 7.42 (2H, m), 7.4-7.25 (8H, m), 6.9 (1H, d, J=7 Hz), 6.6 (1H, d, J=7 Hz), 6.0 (1H, app. t, J=7 Hz), 5.17 (2H, s), 5.11 (2H, s).

### Step 2: 1-Benzyl-3-hydroxypyridine-2(1H)-one (J2)

1-Benzyl-3-(benzyloxy)pyridine-2(1H)-one J1 (1 equivalent) was dissolved in EtOAc and 10% Pd on carbon (0.5 equivalents) and a few drops of glacial acetic acid was added and the reaction stirred at room temperature overnight under an balloon atmosphere of H₂. The crude reaction was filtered through celite and the solvent removed under reduced pressure to give the crude product J2 which was used in the next reaction.

¹H NMR (CDCl₃, 400 MHz,) δ 7.4-7.2 (5H, m), 6.81 (1H, d, J=7 Hz), 6.78 (1H, d, J=7 Hz), 6.12 (1H, app. t, J=7 Hz), 5.17 (2H, s), 5.18 (2H, s).

### Step 3: Methyl 1-benzyl-3-hydroxy-2-oxo-1,2-dihydropyridine-4-carboxylate (J3)

1-Benzyl-3-hydroxypyridine-2(1H)-one J2 (1 equivalent) and K₂CO₃ (5 equivalents) were ground to a fine powder and placed in a round bottom flask under high vacuum. The flask was heated to 60° C. for 24 hrs and remained on the vacuum pump for 5 days. The sample was then placed in a Parr high pressure vessel with was purged with carbon dioxide three times, pressurized to 900 psi and heated to 180° C. The reaction was allowed to proceed for 3 days, then cooled to room temperature and the pressure released. The crude solid was suspended in MeOH and the K₂CO₃ filtered off. The solution was concentrated and the crude material was dissolved in MeOH and treated with thionyl chloride (4 equivalents), then refluxed overnight. The reaction was evaporated to dryness, evaporated from DCM three times, then purified by reverse phase chromatography to give the product J3.

¹H NMR (CDCl₃, 400 MHz,) δ 7.3 (5H, m), 6.8 (1H, d, J=7.3 Hz), 6.4 (1H, d, J=7.3 Hz), 5.14 (2H, s), 3.92 (3H, s).

### Step 4: 1-Benzyl-N-(2,3-dimethoxybenzyl)-3-hydroxy-2-oxo-1,2-dihydropyridine-4-carboxamide (J4)

The methyl ester J3 (1 equivalent) was heated at 100° C. in 2,3-dimethoxybenzylamine (35 equivalents) overnight. The reaction was cooled, diluted with water and extracted with DCM. The organic phase was dried, concentrated and the residue purified by reverse phase chromatography twice to give the desired amide J4.

¹H NMR (CDCl₃, 400 MHz,) δ 8.1 (1H, br. s), 7.3 (3H, m), 7.25 (2H, m), 7.0 (2H, m), 6.95 (1H, m), 6.83 (3H, m), 5.14 (2H, s), 4.64 (2H, d, J=5.6 Hz), 3.88 (3H, s), 3.84 (3H, s).

### EXAMPLE 11

### N²-Benzyl-N⁴-(4-fluorobenzyl)-5,6-dihydroxy-N²-methylpyridine-2,4-dicarboxamide

### Step 1: 5,6-bis(Benzyloxy)-4-{[(4-fluorobenzyl)amino]-carbonyl}-pyridine-2-carboxylic acid (K1)

To a stirred solution of 2,3-bis(benzyloxy)-N-(4-fluorobenzyl)-6-iodoisonicotinamide I1 (1 equivalent) in DMF/H₂O (1:1) under a balloon of CO was added K₂CO₃ (4 equivalents) and palladium(II) acetate (4 mol %). This stirred for 18 h at ambient temperature. Water was added and the resulting precipitate was filtered off. The filtrate was concentrated in vacuo and the residue dissolved in a minimum amount of MeOH and water. The pH was adjusted to 7 by addition of saturated NH₄Cl solution and the resulting precipitate collected by filtration and dried under vacuum to afford the acid K1 as a white solid. HPLC RT=3.42 min, [Hewlett-Packard Zorbax SB-C₈ column, λ=215 nm, 95% H₂O/MeCN to 5% H₂O/MeCN (+0.1% TFA) over 4.5 min, Flow=3.0 mL/min]; LC-MS C₂₈H₂₃FN₂O₅ requires: 486, found: 487 (M+H⁺).

### Step 2: N²-benzyl-5,6-bis(benzyloxy)-N⁴-(4-fluorobenzyl)-N²-methylpyridine-2,4-dicarboxamide (K2)

To a solution of the acid K1 (1 equivalent), N-methyl benzylamine (1 equivalent), and HOBT.H₂O (1.1 equivalents) in DMF was added EDC (1.5 equivalents). Diisopropylethylamine was then added in portions to bring the pH of the solution to 6-7 as measured on wetted E. Merck pH indicator strips. The mixture was stirred at ambient temperature for 18 h, and the solvent was removed under reduced pressure. The residue was purified directly by filtration through a plug of silica gel using 5%, 10%, then 15% MeOH in DCM as eluents. Desired fractions were concentrated in vacuo to afford the amide K2 as a viscous yellow oil. HPLC RT=3.80 min, [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₃₆H₃₂FN₃O₄ requires: 589, found: 590 (M+H⁺).

### Step 3: N²-Benzyl-N⁴-(4-fluorobenzyl)-5,6-dihydroxy-N²-methylpyridine-2,4-dicarboxamide (K3)

To a solution of the amide K2 (1 equivalent) in EtOH at ambient temperature was added Palladium black. This was stirred under a balloon of H₂ for 4 h and then filtered through a bed of celite. The filtrate solution was concentrated in vacuo to give the desired dihydroxypyridine K3. HPLC RT=2.81 min, [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₂₂H₂₀FN₃O₄ requires: 409, found: 410 (M+H⁺).

### EXAMPLE 12

### N²-Benzyl-N⁴-(4-fluorobenzyl)-5-hydroxy-N²,1-dimethyl-6-oxo-1,6-dihydropyridine-2,4-dicarboxamide

### Step 1: N²-benzyl-N⁴-(4-fluorobenzyl)-5-methoxy-N²,1-dimethyl-6-oxo-1,6-dihydropyridine-2,4-dicarboxamide (L1)

To a solution of N²-benzyl-N⁴-(4-fluorobenzyl)-5,6-dihydroxy-N²-methylpyridine-2,4-dicarboxamide K3 (1 equivalent) in THF at ambient temperature was added methyl iodide (5 equivalents) and cesium carbonate (2 equivalents). This was refluxed for 6 h and stirred for 72 h at ambient temperature. The mixture was concentrated in vacuo and the residue partitioned between EtOAc and water. The organic layer was separated, dried (Na₂SO₄), and concentrated in vacuo. The residue was purified by reverse phase HPLC and the desired fractions were concentrated in vacuo to give L1 as a viscous yellow oil. HPLC RT=2.92 min, [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₂₄H₂₄FN₃O₄ requires: 437, found: 438 (M+H⁺).

### Step 2: N²-Benzyl-N⁴-(4-fluorobenzyl)-5-hydroxy-N²,1-dimethyl-6-oxo-1,6-dihydropyridine-2,4-dicarboxamide (L2)

To a solution of the 5-methoxy-1-methylpyridine L1 (1 equivalent) in DCM at 0° C. under N₂ atmosphere was added boron tribromide (5 equivalents). This was allowed to equilibrate to ambient temperature over 3 h and quenched with MeOH. The mixture was concentrated in vacuo and purified by reverse phase HPLC. Desired fractions were concentrated in vacuo to give the desired N-methylpyridine L2 as a pink amorphous solid. HPLC RT=2.88 min, [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₂₃H₂₂FN₃O₄ requires: 423, found: 424 (M+H⁺); ¹H NMR (400 MHz, CD₃OD) Major rotamer: δ 7.3-7.4 (6H, m), 7.15 (1H, d, J=7.14 Hz), 7.03 (2H, t, J=8.79 Hz), 6.71 (1H, s), 4.72 (2H, s), 4.58 (1H, br. s), 4.54 (2H, s), 4.52 (1H, s), 3.48 (3H, s), 2.92 (3H, s).

### EXAMPLE 13

### N-(4-Fluorobenzyl)-2,3-dihydroxy-6-{[4-(morpholin-4-ylmethyl)piperidin-1-yl]carbonyl}isonicotinamide

To a solution of 5,6-bis(benzyloxy)-4-{[(4-fluorobenzyl)amino]-carbonyl}-pyridine-2-carboxylic acid K1 (1 equivalent), 4-(4-morpholinomethyl)piperidine hydrochloride (1 equivalent), and HOBT hydrate (1.2 equivalents) in DMF was added EDC (1.5 equivalents). Diisopropylethylamine was then added in portions to bring the pH of the solution to 6-7 as measured on wetted E. Merck pH indicator strips. The mixture was stirred at ambient temperature for 18 h, and then the solvent was removed under reduced pressure. The residue was dissolved in HOAc and 30% HBr in HOAc was added. The mixture was stirred at ambient temperature for 30 min and the solvent was removed under reduced pressure. The residue was purified by preparative reverse phase HPLC, the desired fractions were combined and evaporated to dryness in vacuo to give the TFA salt of the title compound as an amorphous solid. HPLC RT=2.16 min, [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₂₄H₂₉FN₄O₅ requires: 472, found: 473 (M+H⁺).

### EXAMPLE 14

### N-(4-Fluorobenzyl)-2,3-dihydroxy-6-(trifluoromethyl)isonicotinamide

To a stirred solution of 2,3-bis(benzyloxy)-N-(4-fluorobenzyl)-6-iodoisonicotinamide I1 (1 equivalent) in pyridine at −78° C. was condensed iodotrifluoromethane (ca. 30 equivalents). Copper powder (1 equivalent) was added and the reaction vessel was sealed and heated in a microwave oven at 150° C. for 30 min. The solvents were removed in vacuo and the residue was purified by preparative reverse phase HPLC, the desired fractions were combined and evaporated to dryness in vacuo to give the title compound as an amorphous solid. HPLC RT=2.76 min, [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₁₄H₁₀F₄N₂O₃ requires: 330, found: 331 (M+H⁺).

### EXAMPLE 15

### N-(4-Fluorobenzyl)-2,3-dihydroxy-6-pyrimidin-5-ylisonicotinamide

To a solution of 2,3-bis(benzyloxy)-N-(4-fluorobenzyl)-6-iodoisonicotinamide I1 (1 equivalent) in DMF was added pyrimidine-5-boronic acid (1.3 equivalents), the stirred solution was degassed by bubbling N₂ through it. Cesium carbonate (1.3 equivalents) was added followed by bis-(tri-tert-butylphosphine)palladium (15 mol %). The mixture was heated with stirring in a microwave oven at 120° C. for 40 min. The mixture was purified by preparative reverse phase HPLC and the desired fractions were combined and the solvent was removed in vacuo. The residue was dissolved in 30% HBr in HOAc. After 5 min, the reaction was complete and the solvents were removed in vacuo. The residue was purified by preparative reverse phase HPLC and the desired fractions were combined and the solvent was removed in vacuo to give the TFA salt of the title compound. HPLC RT=2.59 min [Hewlett-Packard Zorbax SB-C8 column, λ=215 nm, 95% H₂O/MeCN to 5% H₂O/MeCN (+0.1% TFA) over 4.5 min, Flow=3.0 mL/min]; LC-MS C₁₇H₁₃FN₄O₃ requires: 340, found: 341 (M+H⁺).

Table 1 below lists compounds of the present invention which have been prepared.

The table provides the structure and name of each compound, the mass of its molecular ion plus 1 (M⁺) or molecular ion minus 1 (M⁻) as determined via FIA-MS or ES, and a reference to the preparative example that is or is representative of the procedure employed to prepare the compound.

| Structure | Name | Ex | M+H⁺ |
|---|---|---|---|
| 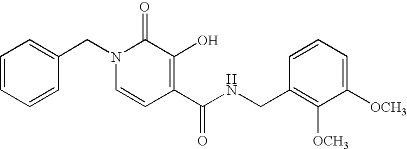 | 1-benzyl-N-(2,3-dimethoxybenzyl)- 3-hydroxy-2-oxo-1,2- dihydropyridine-4-carboxamide | 10 | 395 |
| 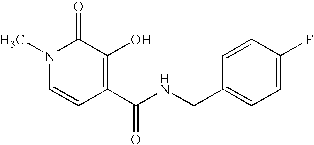 | N-(4-Fluorobenzyl)-3-hydroxy-1- methyl-2-oxo-1,2-dihydropuridine- 4-carboxamide | 8 | 277 |
| 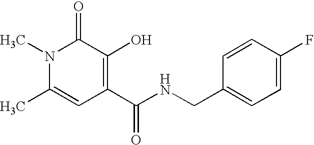 | N-(4-Fluorobenzyl)-3-hydroxy-1,6- dimethyl-2-oxo-1,2- dihydropyridine-4-carboxamide | 12 | 291 |
| 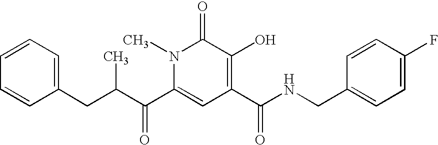 | N²-Benzyl-N⁴-(4-fluorobenzyl)-5- hydroxy-N²,1-dimethyl-6-oxo-1,6- dihydropyridine-2,4-dicarboxamide | 12 | 424 |
| 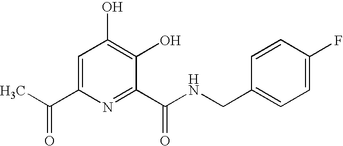 | 6-Acetyl-N-(4-fluorobenzyl)-3,4- dihydroxypyridine-2-carboxamide | 1 | 305 |
| 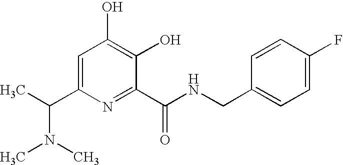 | 6-[1-(Dimethylamino)ethyl]-N-(4- fluorobenzyl)-3,4-dihydroxy- pyridine-2-carboxamide | 7 | 334 |
| 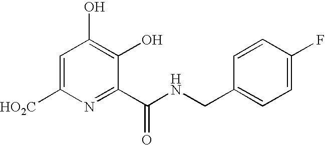 | 6-{[(4-Fluorobenzyl)amino]- carbonyl}-4,5-dihydroxypyridine-2- carboxylic acid | 2 | 307 |
| 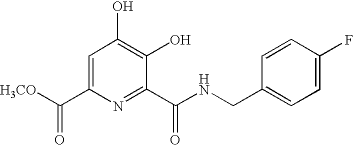 | Methyl 6-{[(4-fluorobenzyl)amino]- carbonyl}-4,5-dihydroxypyridine-2- carboxylate | 3 | 321 |
| 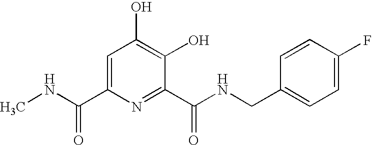 | N²-(4-Fluorobenzyl)3,4-dihydroxy- N⁶-methylpyridine-2,6- dicarboxamide | 4 | 320 |
| 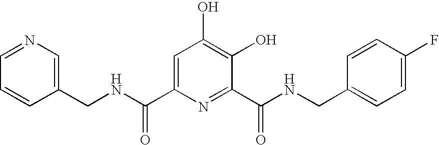 | N²-(4-Fluorobenzyl)-3,4-dihydroxy- N⁶-(pyridin-3-ylmethyl)pyridine- 2,6-dicarboxamide | 4 | 395 (M−H⁻) |
| 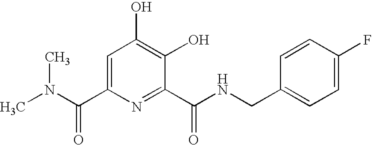 | N²-(4-Fluorobenzyl)-3,4-dihydroxy- N⁶,N⁶-dimethylpyridine-2,6- dicarboxamide | 4 | 334 |
| 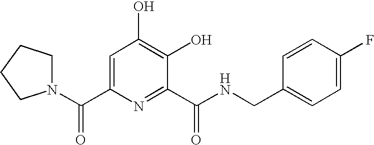 | N-(4-Fluorobenzyl)-3,4-dihydroxy- 6-pyrrolidin-1-ylcarbonyl-pyridine- 2-carboxamide; | 4 | 360 |
| 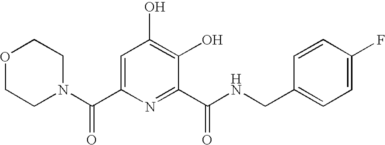 | N-(4-Fluorobenzyl)-3,4-dihydroxy- 6-(morpholin-4-ylcarbonyl)- pyridine-2-carboxamide | 4 | 376 |
| 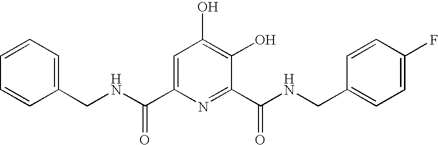 | N⁶-Benzyl-N²-(4-fluorobenzyl)-3,4- dihydroxypyridine-2,6- dicarboxamide | 4 | 396 |
| 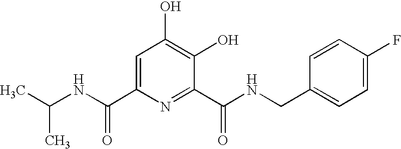 | N²-(4-Fluorobenzyl)-3,4-dihydroxy- N⁶-isopropylpyridine-2,6- dicarboxamide | 4 | 362 |
| 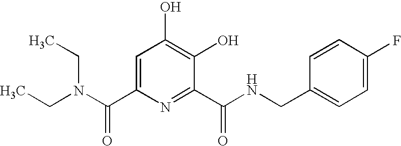 | N²-(4-Fluorobenzyl)-3,4-dihydroxy- N⁶,N⁶-diethylpyridine-2,6- dicarboxamide | 4 | 348 |
| 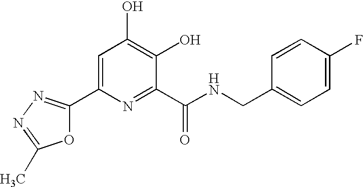 | N-(4-Fluorobenzyl)-3,4-dihydroxy- 6-((5-methyl)-1,3,4-oxadiazol-2-yl)- pyridine-2-carboxamide | 5 | 345 |
| 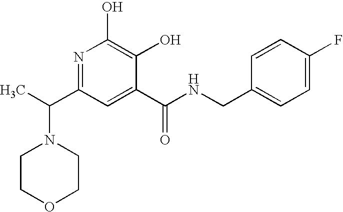 | N-(4-Fluorobenzyl)-2,3-dihydroxy- 6-[1-(morpholin-4-yl)ethyl]-4- pyridinecarboxamide | 9 | 376 |
| 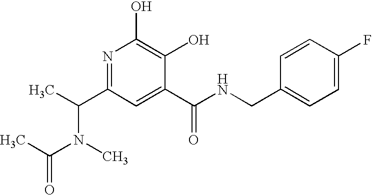 | 6-{1-[Acetyl(methyl)amino]ethyl}- N-(4-fluorobenzyl)-2,3- dihydroxyisonicotinamide | 9 | 362 |
| 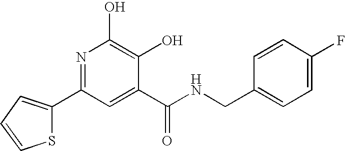 | N-(4-Fluorobenzyl)-2,3-dihydroxy- 6-(2-thienyl)-4-pyridine- carboxamide | 6 | 345 |
| 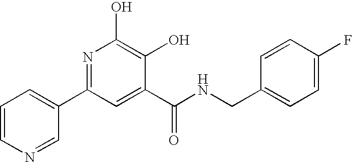 | N-(4-Fluorobenzyl)-2,3-dihydroxy- 6-(3-pyridinyl)-4-pyridine- carboxamide | 6 | 340 |
| 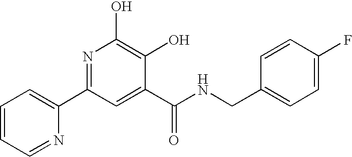 | N-(4-Fluorobenzyl)-2,3-dihydroxy- 6-(2-pyridinyl)-4-pyridine- carboxamide | 6 | 340 |
| 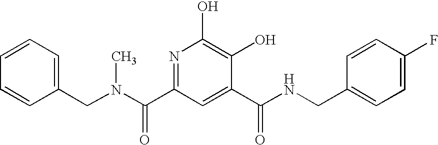 | N²-Benzyl-N⁴-(4-fluorobenzyl)-5,6- dihydroxy-N²-methylpyridine-2,4- dicarboxamide | 11 | 410 |
| 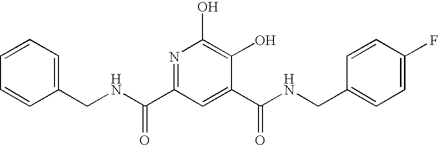 | N²-benzyl-N⁴-(4-fluorobenzyl)-5,6- dihydroxypyridine-2,4- dicarboxamide | 13 | 396 |
| 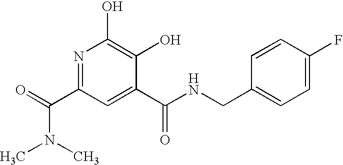 | N⁴-(4-Fluorobenzyl)-5,6-dihydroxy- N²,N²-dimetyhylpyridine-2,4- dicarboxamide | 13 | 334 |
| 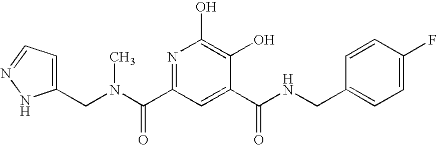 | N⁴-(4-Fluorobenzyl)-5,6-dihydroxy- N²-methyl-N²-(1H-pyrazol-5- ylmethyl)pyridine-2,4- dicarboxamide | 13 | 400 |
| 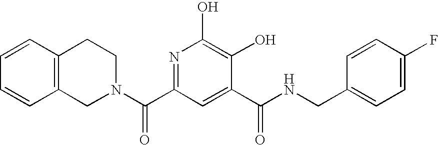 | 6-(3,4-Dihydroxyisoquinolin-2(1H)- ylcarbonyl)-N-(4-fluorobenzyl)-2,3- dihydroxyisonicotinamide | 13 | 422 |
| 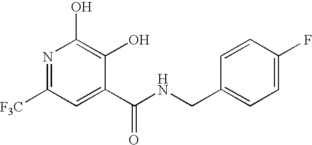 | N-(4-Fluorobenzyl)-2,3-dihydroxy- 6-(trifluoromethyl)isonicotinamide | 14 | 331 |
| 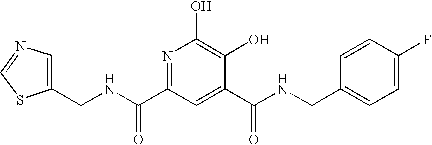 | N⁴-(4-Fluorobenzyl)-5,6-dihydroxy- N²-(1,3-thiazol-5-ylmethyl)- pyridine-2,4-dicarboxamide | 13 | 403 |
| 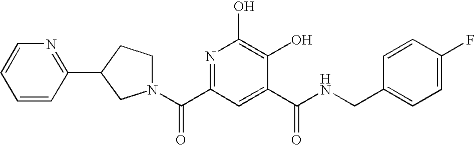 | N-(4-Fluorobenzyl)-2,3-dihydroxy- 6-[(3-pyridin-2-ylpyrrolidin-1- yl)carbonyl]isonicotinamide | 13 | 427 |
| 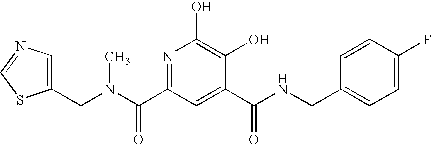 | N⁴-(4-Fluorobenzyl)-5,6-dihydroxy- N²-methyl-N²-(1,3-thiazol-5- ylmethyl)pyridine-2,4- dicarboxamide | 13 | 417 |
| 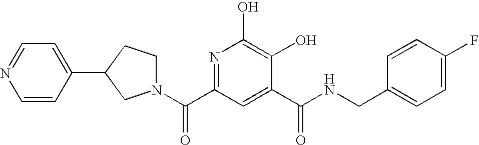 | N-(4-Fluorobenzyl)-2,3-dihydroxy- 6-[(3-pyridin-4-ylpyrrolidin-1- yl)carbonyl]isonicotinamide | 13 | 437 |
| 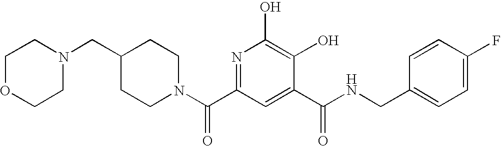 | N-(4-Fluorobenzyl)-2,3-dihydroxy- 6-{[4-(morpholin-4-ylmethyl)piperi- din-1-yl]carbonyl}isonicotinamide | 13 | 473 |
| 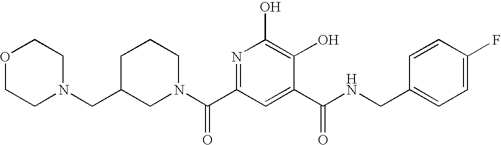 | N-(4-Fluorobenzyl)-2,3-dihydroxy- 6-{[3-(morpholin-4-ylmethyl)piperi- din-1-yl]carbonyl}isonicotinamide | 13 | 437 |
| 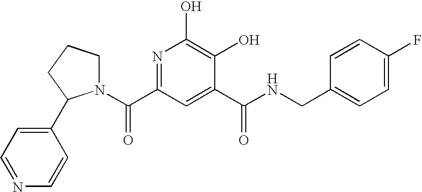 | N-(4-Fluorobenzyl)-2,3-dihydroxy- 6-[(2-pyridin-4-ylpyrrolidin-1- yl)carbonyl]isonicotinamide | 13 | 437 |
| 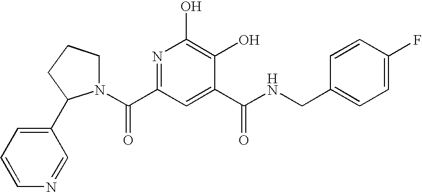 | N-(4-Fluorobenzyl)-2,3-dihydroxy- 6-[(2-pyridin-3-ylpyrrolidin-1- yl)carbonyl]isonicotinamide | 13 | 437 |
| 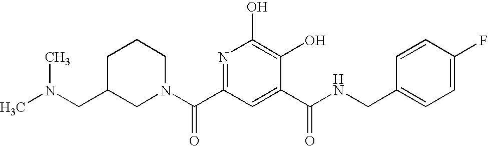 | 6-({3-[(Dimethylamino)methyl]- piperidin-1-yl}carbonyl)-N-(4- fluorobenzyl)-2,3- dihydroxyisonicotinamide | 13 | 431 |
| 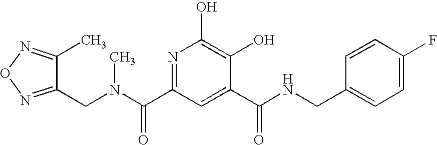 | N⁴-(4-Fluorobenzyl)-5,6-dihydroxy- N²-methyl-N²-[(4-methyl-1,2,5- oxadiazol-3-yl)methyl]pyridine-2,4- dicarboxamide | 13 | 416 |
| 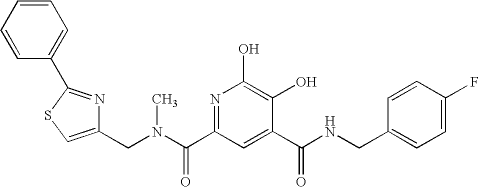 | N⁴-(4-Fluorobenzyl)-5,6-dihydroxy- N²-methyl-N²-[(2-phenyl-1,3- thiazol-4-yl)methyl]pyridine-2,4- dicarboxamide | 13 | 493 |
| 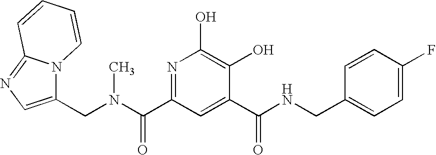 | N⁴-(4-Fluorobenzyl)-5,6-dihydroxy- N²-(imidazo[1,2-a]pyridin-3- ylmethyl)-N²-methylpyridine-2,4- dicarboxamide | 13 | 450 |
| 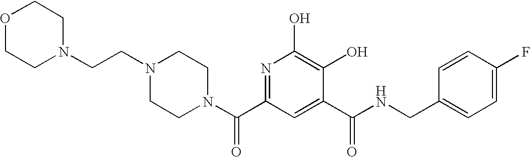 | N-(4-Fluorobenzyl)-2,3-dihydroxy- 6-{[4-(2-morpholin-4-ylethyl)- piperazin-1-yl]carbonyl}- isonicotinamide | 13 | 488 |
| 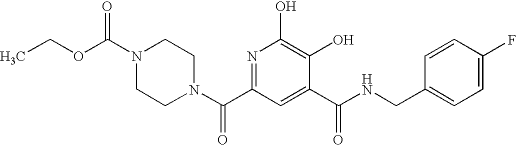 | Ethyl 4-[(4-{[4-(fluorobenzyl)- amino]carbonyl}-5,6-dihydroxy- pyridin-2-yl)carbonyl]piperazine-1- carboxylate | 13 | 447 |
| 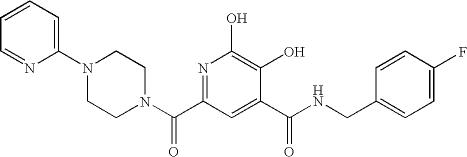 | N-(4-Fluorobenzyl)-2,3-dihydroxy- 6-[(4-pyridin-2-ylpiperazin-1- yl)carbonyl]isonicotinamide | 13 | 453 |
| 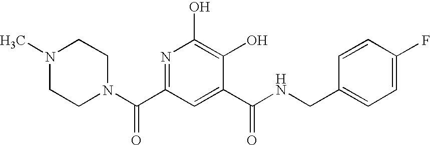 | N-(4-Fluorobenzyl)-2,3-dihydroxy- 6-[(4-methylpiperazin-1- yl)carbonyl]isonicotinamide | 13 | 389 |
| 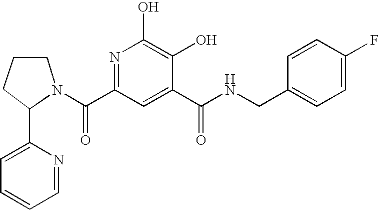 | N-(4-Fluorobenzyl)-2,3-dihydroxy- 6-[(2-pyridin-2-ylpyrrolidin-1- yl)carbonyl]isonicotinamide | 13 | 437 |
| 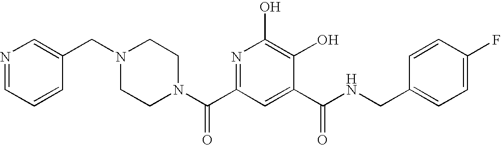 | N-(4-Fluorobenzyl)-2,3-dihydroxy- 6-{[4-(pyridin-3-ylmethyl)pipera- zin-1-yl]carbonyl}isonicotinamide | 13 | 466 |
| 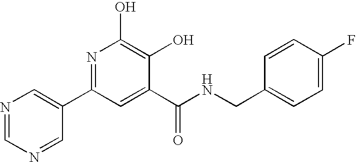 | N-(4-Fluorobenzyl)-2,3-dihydroxy- 6-pyrimidin-5-ylisonicotinamide | 15 | 341 |
| 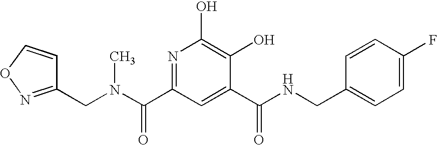 | N⁴-(4-Fluorobenzyl)-5,6-dihydroxy- N²-(isoxazol-3-ylmethyl)-N²- methylpyridine-2,4-dicarboxamide | 13 | 401 |
| 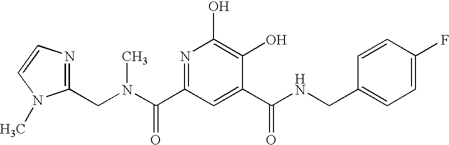 | N⁴-(4-Fluorobenzyl)-5,6-dihydroxy- N²-methyl-N²-[(1-methyl-1H- imidazol-2-yl)methyl]pyridine-2,4- dicarboxamide | 13 | 414 |
| 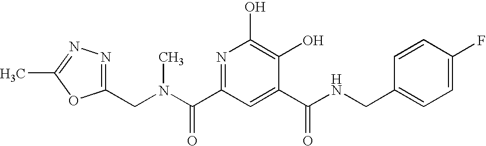 | N⁴-(4-Fluorobenzyl)-5,6-dihydroxy- N²-methyl-N²-[(5-methyl-1,3,4- oxadiazol-2-yl)methyl]pyridine-2,4- dicarboxamide | 13 | 416 |
| 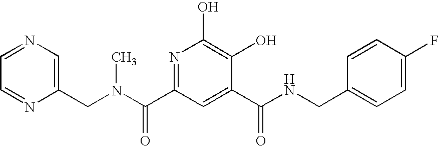 | N⁴-(4-Fluorobenzyl)-5,6-dihydroxy- N²-methyl-N²-(pyrazin-2-ylmethyl)- pyridine-2,4-dicarboxamide | 13 | 412 |

While the foregoing specification teaches the principles of the present invention, with examples provided for the purpose of illustration, the practice of the invention encompasses all of the usual variations, adaptations and/or modifications that come within the scope of the following claims.

1. A compound of Formula I, or a pharmaceutically acceptable salt thereof: 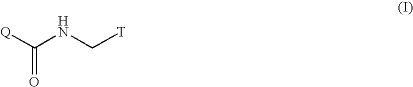
wherein:
Q is: 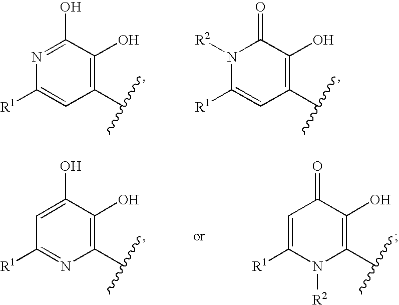
T is: 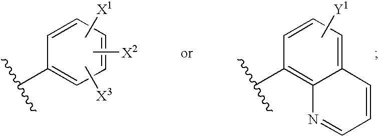
X¹, X² and X³ are each independently selected from the group consisting of —H, halo, —C₁₋₄ alkyl, —O—C₁₋₄ alkyl, —C₁₋₄ fluoroalkyl, —SO₂—C₁₋₄ alkyl, —C(═O)—NH(—C₁₋₄ alkyl), —C(═O)—N(—C₁₋₄ alkyl)₂, and HetA
Y¹ is —H, halo, —C₁₋₄ alkyl, or —C₁₋₄ fluoroalkyl;
R¹ is:
(1) —C₁₋₆ fluoroalkyl,
(2) —C₁₋₆ alkyl-N(R^{a})R^{b},
(3) —C₁₋₆ alkyl-N(R^{a})—C(═O)—R^{b},
(4) —C(═O)—R^{a},
(5) —C(═O)OR^{a},
(6) —C(═O)—N(R^{a})R^{b},
(7) —C(═O)—N(R^{a})—C₁₋₆ alkyl-aryl,
(8) —HetB,
(9) —C(═O)—N(R^{a})—C₁₋₆ alkyl—HetB,
(10) —C₁₋₆ alkyl-HetC,
(11) —C(═O)—HetC.
(12) —C(═O)-aryl, or
(13) —C(═O)—HetB;
each HetA is independently a 5- or 6-membered heteroaromatic ring containing from 1 to 4 heteroatoms independently selected from N, O and S, wherein the heteroaromatic ring is optionally substituted with 1 or 2 substituents each of which is independently a —C₁₋₄ alkyl;
HetB is:
(A) a 5- or 6-membered heteroaromatic ring containing from 1 to 4 heteroatoms independently selected from N, O and S; wherein the heteroaromatic ring is attached to the rest of the compound via a carbon atom in the ring, and wherein the heteroaromatic ring is:
(i) optionally substituted with 1 or 2 substituents each of which is independently a —C₁₋₄ alkyl; and
(ii) optionally substituted with aryl or —C₁₋₄ alkyl-aryl; or
(B) a 9- or 10-membered aromatic heterobicyclic fused ring system containing from 1 to 4 heteroatoms independently selected from N, O and S; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
(i) optionally substituted with 1 or 2 substituents each of which is independently a —C₁₋₄ alkyl; and
(ii) optionally substituted with aryl or —C₁₋₄ alkyl-aryl;
HetC is a 4- to 7-membered saturated heterocyclic ring containing at least one carbon atom and a total of from 1 to 4 heteroatoms independently selected from 1 to 4 N atoms, from 0 to 2 O atoms, and from 0 to 2 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with 1 or 2 substituents each of which is independently a —C₁₋₄ alkyl, —C₁₋₄ alkyl-N(R^{a})R^{b}, or —C(═O)OR^{a}; and
(ii) optionally substituted with aryl, —C₁₋₄ alkyl-aryl, HetD, or —C₁₋₄ alkyl-HetD; wherein HetD is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S;
R² is —C₁₋₆ alkyl or —C₁₋₆ alkyl-aryl;
aryl is phenyl or naphthyl;
each R^{a} is independently H or C₁₋₆ alkyl; and
each R^{b} is independently H or C₁₋₆ alkyl.

2. The compound according to claim 1, or a pharmaceutically acceptable salt thereof, wherein
R¹ is:
(1) —C₁₋₃ fluoroalkyl,
(2) —C₁₋₃ alkyl-NH₂.
(3) —C₁₋₃ alkyl-NH(—C₁₋₃ alkyl),
(4) —C₁₋₃ alkyl-N(—C₁₋₃ alkyl)₂,
(5) —C₁₋₃ alkyl-NH—C(═O)—C₁₋₃ alkyl
(6) —C₁₋₃ alkyl-N(—C₁₋₃ alkyl)-C(═O)—C₁₋₃ alkyl,
(7) —C(═O)H,
(8) —C(═O)—C₁₋₃ alkyl.
(9) —CO₂H,
(10) —C(═O)O—C₁₋₃ alkyl,
(11) —C(═O)—NH(—C₁₋₃ alkyl),
(12) —C(═O)—N(—C₁₋₃ alkyl)₂,
(13) —C(═O)—NH—CH₂-phenyl,
(14) —C(═O)—N(CH₃)—CH₂-phenyl,
(15) —HetB,
(16) —C(═O)—NH—CH₂—HetB,
(17) —C(═O)—N(CH₃)—CH₂—HetB,
(18) —CH₂—HetC,
(19) —CH(CH₃)—HetC, or
(20) —C(═O)—HetC;
HetB is:
(A) a 5- or 6-membered heteroaromatic ring containing a total of from 1 to 3 heteroatoms independently selected from zero to 3 N atoms, zero or 10 atoms, and zero or 1 S atoms; wherein the heteroaromatic ring is attached to the rest of the compound via a carbon atom in the ring, and wherein the heteroaromatic ring is:
(i) optionally substituted with 1 or 2 substituents each of which is independently a —C₁₋₃ alkyl; and
(ii) optionally substituted with phenyl or —CH₂-phenyl; or
(B) a 9- or 10-membered aromatic heterobicyclic fused ring system containing a total of from 1 to 4 hetero atoms independently selected from 1 to 4 N atoms, zero or 1 o atoms, and zero or I S atoms; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
(i) optionally substituted with 1 or 2 substituents each of which is independently a —C₁₋₃ alkyl; and
(ii) optionally substituted with phenyl or —CH₂-phenyl; and
HetC is a 5- or 6-membered saturated heterocyclic ring containing a total of from 1 to 3 heteroatoms independently selected from 1 to 3 N atoms, zero or 1 O atoms, and zero or 1 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with —C₁₋₃ alkyl, —(CH₂)₁₋₂—NH(—C₁₋₃ alkyl), —(CH₂)₁₋₂—N(—C₁₋₃ alkyl)₂ or —C(═O)O—C₁₋₃ alkyl; and
(ii) optionally substituted with phenyl, —CH₂-phenyl, HetD, or —(CH₂)₁₋₂—HetD; wherein HetD is (i) a 5- or 6-membered heteroaromatic ring containing a total of from 1 to 3 heteroatoms independently selected from zero to 3 N atoms, zero or 1 O atoms, and zero or 1 S atoms or (ii) a 5- or 6-membered saturated heterocyclic ring containing a total of from 1 to 3 heteroatoms independently selected from 1 to 3 N atoms, zero or 1 O atoms, and zero or 1 S atoms.

3. The compound according to claim 2, or a pharmaceutically acceptable salt thereof, wherein R¹ is:
(1) —CF₃,
(2) —CH(CH₃)—N(CH₃)₂,
(3) —C(═O)—CH₃,
(4) —CO₂H,
(5) —C(═O)OCH₃,
(6) —C(═O)—NH(CH₃),
(7) —C(═O)—N(CH₃)₂,
(8) —C(═O)—NH(CH₂CH₃),
(9) —C(═O)—N(CH₂CH₃)₂,
(10) —C(═O)—NH(CH(CH₃)₂),
(11) —C(═O)—NH—CH₂-phenyl,
(12) —C(═O)—N(CH₃)—CH₂-phenyl,
(13) —HetB,
(14) —C(═O)—NH—CH₂—HetB,
(15) —C(═O)—N(CH₃)—CH₂—HetB, or
(16) —C(═O)—HetC;
HetB is a heteroaromatic ring selected from the group consisting of oxadiazolyl, thiophenyl, pyrazolyl, thiazolyl, isothiazolyl, oxazolyl, isoxazolyl, imidazolyl, pyridinyl, pyrimidinyl, pyrazinyl, and pyridoimidazolyl; wherein the heteroaromatic ring is attached to the rest of the compound via a carbon atom in the ring, and wherein the heteroaromatic ring is optionally substituted with methyl or phenyl; and
HetC is a heterocyclic ring selected from the group consisting of pyrrolidinyl, morpholinyl, piperidinyl, piperazinyl, and piperidinyl fused with a benzene ring; wherein the heterocyclic ring is attached to the rest of the compound via a N atom in the ring, and wherein the heterocyclic ring is optionally substituted with methyl, —CH₂N(CH₃)₂, —C(═O)OCH₂CH₃, pyridinyl, —CH₂-pyridinyl, —CH₂-morpholinyl, or —CH₂CH₂-morpholinyl.

4. The compound according to claim 1, or a pharmaceutically acceptable salt thereof, wherein T is 4-fluorophenyl.

5. The compound according to claim 1, or a pharmaceutically acceptable salt thereof, wherein R² is methyl.

6. A compound according to claim 1, or a pharmaceutically acceptable salt thereof, which is a compound selected from the group consisting of:
N²-benzyl-N²-(4-fluorobenzyl)-5-hydroxy-N², 1-dimethyl-6-oxo-1,6-dihydropyridine-2,4-dicarboxamide;
6-acetyl-N-(4-fluorobenzyl)-3,4-dihydroxypyridine-2-carboxamide;
6-[1-(dimethylamino)ethyl]-N-(4-fluorobenzyl)-3,4-dihydroxypyridine-2-carboxamide;
6-{[(4-fluorobenzyl)amino]carbonyl}-4,5-dihydroxypyridine-2-carboxylic acid;
methyl 6-{[(4-fluorobenzyl)amino]carbonyl}-4,5-dihydroxypyridine-2-carboxylate;
N²-(4-fluorobenzyl)-3,4-dihydroxy-N⁶-methylpyridine-2,6-dicarboxamide;
N²-(4-fluorobenzyl)-3,4-dihydroxy-N⁶-(pyridin-3-ylmethyl)pyridine-2,6-dicarboxamide;
N²-(4-fluorobenzyl)-3,4-dihydroxy-N⁶,N⁶-dimethylpyridine-2,6-dicarboxamide;
N-(4-fluorobenzyl)-3,4-dihydroxy-6-pyrrolidin-1-ylcarbonyl-pyridine-2-carboxamide;
N-(4-fluorobenzyl)-3,4-dihydroxy-6-(morpholin-4-ylcarbonyl)-pyridine-2-carboxamide;
N⁶-Benzyl-N²-(4-fluorobenzyl)-3,4-dihydroxypyridine-2,6-dicarboxamide;
N²-(4-fluorobenzyl)-3,4-dihydroxy-N⁶-isopropylpyridine-2,6-dicarboxamide;
N²-(4-fluorobenzyl)-3,4-dihydroxy-N⁶,N⁶-diethylpyridine-2,6-dicarboxamide;
N-(4-fluorobenzyl)-3,4-dihydroxy-6-((5-methyl)-1,3,4-oxadiazol-2-yl)-pyridine-2-carboxamide;
N-(4-fluorobenzyl)-2,3-dihydroxy-6-[1-(morpholin-4-yl)ethyl]-4-pyridinecarboxamide;
6-{1-[acetyl(methyl)amino]ethyl}-N-(4-fluorobenzyl)-2,3-dihydroxyisonicotinamide;
N-(4-fluorobenzyl)-2,3-dihydroxy-6-(2-thienyl)-4-pyridinecarboxamide;
N-(4-fluorobenzyl)-2,3-dihydroxy-6-(3-pyridinyl)-4-pyridinecarboxamide;
N-(4-fluorobenzyl)-2,3-dihydroxy-6-(2-pyridinyl)-4-pyridinecarboxamide;
N²-benzyl-N⁴-(4-fluorobenzyl)-5,6-dihydroxy-N²-methylpyridine-2,4-dicarboxamide;
N²-benzyl-N⁴-(4-fluorobenzyl)-5,6-dihydroxypyridine-2,4-dicarboxamide;
N⁴-(4-fluorobenzyl)-5,6-dihydroxy-N²,N²-dimethylpyridine-2,4-dicarboxamide;
N⁴-(4-fluorobenzyl)-5,6-dihydroxy-N²-methyl-N²-(1H-pyrazol-5-ylmethyl)pyridine-2,4-dicarboxamide;
6-(3,4-dihydroisoquinolin-2(1H)-ylcarbonyl)-N-(4-fluorobenzyl)-2,3-dihydroxyisonicotinamide;
N-(4-fluorobenzyl)-2,3-dihydroxy-6-(trifluoromethyl)isonicotinamide;
N⁴-(4-fluorobenzyl)-5,6-dihydroxy-N²-(1,3-thiazol-5-ylmethyl)pyridine-2,4-dicarboxamide;
N-(4-fluorobenzyl)-2,3-dihydroxy-6-[(3-pyridin-2-ylpyrrolidin-1-yl)carbonyl]isonicotinamide;
N⁴-(4-fluorobenzyl)-5,6-dihydroxy-N²-methyl-N²-(1,3-thiazol-5-ylmethyl)pyridine-2,4-dicarboxamide;
N-(4-fluorobenzyl)-2,3-dihydroxy-6-[(3-pyridin-4-ylpyrrolldin-1-yl)carbonyl]isonicotinamide;
N-(4-fluorobenzyl)-2,3-dihydroxy-6-{[4-(morpholin-4-ylmethyl)piperidin-1-yl]carbonyl} isonicotinamide;
N-(4-fluorobenzyl)-2,3-dihydroxy-6-{[3-(morpholin-4-ylmethyl)piperidin-1-yl]carbonyl}isonicotinamide;
N-(4-fluorobenzyl)-2,3-dihydroxy-6-[(2-pyridin-4-ylpyrrolidin-1-yl)carbonyl]isonicotinamide;
N-(4-fluorobenzyl)-2,3-dihydroxy-6-[(2-pyridin-3-ylpyrrolidin-1-yl)carbonyl]isonicotinamide;
6-({3-[(dimethylamino)methyl]piperidin-1-yl}carbonyl)-N-(4-fluorobenzyl)-2,3-dihydroxyisonicotinamide;
N⁴-(4-fluorobenzyl)-5,6-dihydroxy-N²-methyl-N²— [(4-methyl-1,2,5-oxadiazol-3-yl)methyl]pyridine-2,4-dicarboxamide;
N⁴-(4-fluorobenzyl)-5,6-dihydroxy-N²-methyl-N²— [(2-phenyl-1,3-thiazol-4-yl)methyl]pyridine-2,4-dicarboxamide;
N⁴-(4-fluorobenzyl)-5,6-dihydroxy-N²-(imidazo[1,2-a]pyridin-3-ylmethyl)-N²-methylpyridine-2,4-dicarboxamide;
N-(4-fluorobenzyl)-2,3-dihydroxy-6-{[4-(2-morpholin-4-ylethyl)piperazin-1-yl]carbonyl} isonicotinamide;
ethyl 4-[(4-{[(4-fluorobenzyl)amino]carbonyl}-5,6-dihydroxypyridin-2-yl)carbonyl]piperazine-1-carboxylate;
N-(4-fluorobenzyl)-2,3-dihydroxy-6-[(4-pyridin-2-ylpiperazin-1-yl)carbonyl]isonicotinamide;
N-(4-fluorobenzyl)-2,3-dihydroxy-6-[(4-methylpiperazin-1-yl)carbonyl]isonicotinamide;
N-(4-fluorobenzyl)-2,3-dihydroxy-6-[(2-pyridin-2-ylpyrrolidin-1-yl)carbonyl] isonicotinamide;
N-(4-fluorobenzyl)-2,3-dihydroxy-6-{[4-(pyridin-3-ylmethyl)piperazin-1-yl]carbonyl} isonicotinamide;
N-(4-fluorobenzyl)-2,3-dihydroxy-6-pyrimidin-5-ylisonicotinamide;
N⁴-(4-fluorobenzyl)-5,6-dihydroxy-N²-(isoxazol-3-ylmethyl)-N²-methylpyridine-2,4-dicarboxamide;
N⁴-(4-fluorobenzyl)-5,6-dihydroxy-N²-methyl-N²— [(1-methyl-1H-imidazol-2-yl)methyl]pyridine-2,4-dicarboxamide;
N⁴-(4-fluorobenzyl)-5,6-dihydroxy-N²-methyl-N²— [(5-methyl-1,3,4-oxadiazol-2-yl)methyl]pyridine-2,4-dicarboxamide; and
N⁴-(4-Fluorobenzyl)-5,6-dihydroxy-N²-methyl-N²-(pyrazin-2-ylmethyl)pyridine-2,4-dicarboxamide.

7. A compound of Formula II, or a pharmaceutically acceptable salt thereof: 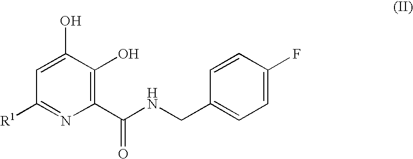
wherein R¹ is:
(1) —C₁₋₄ fluoroalkyl,
(2) —C₁₋₄ alkyl-N(R^{a})R^{b},
(3) —C(═O)—R^{a},
(4) —C(═O)OR^{a},
(5) —C(═O)—N(R^{a})R^{b},
(6) —C(═O)—N(R^{a})—C₁₋₄ alkyl-aryl,
(7) —HetB,
(8) —C(═O)—N(R^{a})—C₁₋₄ alkyl—HetB, or
(9) —C(═O)—HetC;
HetB is:
(A) a 5- or 6-membered heteroaromatic ring containing from 1 to 4 heteroatoms independently selected from N, O and S; wherein the heteroaromatic ring is attached to the rest of the compound via a carbon atom in the ring, and wherein the heteroaromatic ring is:
(i) optionally substituted with 1 or 2 substituents each of which is independently a —C₁₋₄ alkyl; and
(ii) optionally substituted with aryl or —C₁₋₄ alkyl-aryl; or
(B) a 9- or 10-membered aromatic heterobicyclic fused ring system containing from 1 to 4 heteroatoms independently selected from N, O and S; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
(i) optionally substituted with 1 or 2 substituents each of which is independently a —C₁₋₄ alkyl; and
(ii) optionally substituted with aryl or —C₁₋₄ alkyl-aryl;
HetC is a 4- to 7-membered saturated heterocyclic ring containing at least one carbon atom and a total of from 1 to 4 heteroatoms independently selected from 1 to 4 N atoms, from 0 to 2O atoms, and from 0 to 2 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with 1 or 2 substituents each of which is independently a —C₁₋₄ alkyl, —C₁₋₄ alkyl-N(R^{a})R^{b}, or —C(═O)ORₐ; and
(ii) optionally substituted with aryl, —C₁₋₄ alkyl-aryl, HetD, or —C₁₋₄ alkyl-HetD; wherein HetD is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S;
aryl is phenyl or naphthyl;
each R^{a} is independently H or C₁₋₄ alkyl; and
each R^{b} is independently H or C₁₋₄ alkyl.

8. A compound according to claim 7, or a pharmaceutically acceptable salt thereof, wherein R¹ is:
(1) —CF₃,
(2) —C(═O)—CH₃,
(3) —CO₂H,
(4) —C(═O)OCH₃,
(5) —C(═O)—NH(CH₃),
(6) —C(═O)—N(CH₃)₂,
(7) —C(═O)—NH(CH₂CH₃),
(8) —C(═O)—N(CH₂CH₃)₂,
(9) —C(═O)—NH(CH(CH₃)₂),
(10) —C(═O)—NH—CH₂-phenyl,
(11) —C(═O)—N(CH₃)—CH₂-phenyl,
(12) —HetB,
(13) —C(═O)—NH—CH₂—HetB,
(14) —C(═O)—N(CH₃)—CH₂—HetB, or
(15) —C(═O)—HetC.

9. A compound of Formula III, or a pharmaceutically acceptable salt thereof: 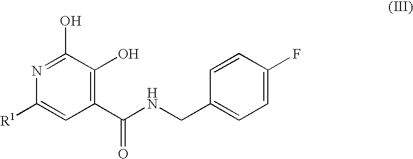
wherein:
R¹ is:
(1) —C₁₋₄ fluoroalkyl,
(2) —C₁₋₄ alkyl-N(R^{a})—C(═O)—R^{b},
(3) —C(═O)—R^{a},
(4) —C(═O)OR^{a},
(5) —C(═O)—N(R^{a})R^{b},
(6) —C(═O)—N(R^{a})—C₁₋₄ alkyl-aryl,
(7) —HetB,
(8) —C(═O)—N(R^{a})—C₁₋₄ alkyl—HetB,
(9) —C₁₋₄ alkyl-HetC, or
(10) —C(═O)—HetC;
HetB is:
(A) a 5- or 6-membered heteroaromatic ring containing from 1 to 4 heteroatoms independently selected from N, O and S; wherein the heteroaromatic ring is attached to the rest of the compound via a carbon atom in the ring, and wherein the heteroaromatic ring is:
(i) optionally substituted with 1 or 2 substituents each of which is independently a —C₁₋₄ alkyl; and
(ii) optionally substituted with aryl or —C₁₋₄ alkyl-aryl; or
(B) a 9- or 10-membered aromatic heterobicyclic fused ring system containing from 1 to 4 heteroatoms independently selected from N, O and S; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
(i) optionally substituted with 1 or 2 substituents each of which is independently a —C₁₋₄ alkyl; and
(ii) optionally substituted with aryl or —C₁₋₄ alkyl-aryl;
HetC is a 4- to 7-membered saturated heterocyclic ring containing at least one carbon atom and a total of from 1 to 4 heteroatoms independently selected from 1 to 4 N atoms, from 0 to 2O atoms, and from 0 to 2 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with 1 or 2 substituents each of which is independently a —C₁₋₄ alkyl, —C₁₋₄ alkyl-N(R^{a})R^{b}, or —C(═O)ORₐ; and
(ii) optionally substituted with aryl, —C₁₋₄ alkyl-aryl, HetD, or —C₁₋₄ alkyl-HetD; wherein HetD is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S;
aryl is phenyl or naphthyl;
R^{a} is H or C₁₋₄ alkyl; and
R^{b} is H or C₁₋₄alkyl.

10. A compound according to claim 9, or a pharmaceutically acceptable salt thereof, wherein R¹ is:
(1) —CF₃,
(2) —C(═O)—CH₃,
(3) —CO₂H,
(4) —C(═O)OCH₃,
(5) —C(═O)—NH(CH₃),
(6) —C(═O)—N(CH₃)₂,
(7) —C(═O)—NH(CH₂CH₃),
(8) —C(═O)—N(CH₂CH₃)₂,
(9) —C(═O)—NH(CH(CH₃)₂),
(10) —C(═O)—NH—CH₂-phenyl,
(11) —C(═O)—N(CH₃)—CH₂-phenyl,
(12) —HetB,
(13) —C(═O)—NH—CH₂—HetB,
(14) —C(═O)—N(CH₃)—CH₂—HetB, or
(15) —C(═O)—HetC.

11. A compound of Formula IV, or a pharmaceutically acceptable salt thereof: 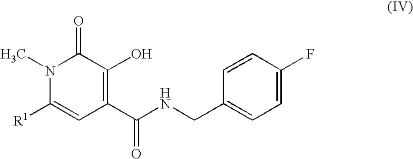
wherein R¹ is:
(1) —C₁₋₄ fluoroalkyl,
(2) —C(═O)—R^{a},
(3) —C(═O)OR^{a},
(4) —C(═O)—N(R^{a})R_{b},
(5) —C(═O)—N(R^{a})—C₁₋₄ alkyl-aryl,
(6) —HetB,
(7) —C(═O)—N(R^{a})—C₁₋₄ alkyl—HetB, or
(8) —C(═O)—HetC;
HetB is:
(A) a 5- or 6-membered heteroaromatic ring containing from 1 to 4 heteroatoms independently selected from N, O and S; wherein the heteroaromatic ring is attached to the rest of the compound via a carbon atom in the ring, and wherein the heteroaromatic ring is:
(i) optionally substituted with 1 or 2 substituents each of which is independently a —C₁₋₄ alkyl; and
(ii) optionally substituted with aryl or —C₁₋₄ alkyl-aryl; or
(B) a 9- or 10-membered aromatic heterobicyclic fused ring system containing from 1 to 4 heteroatoms independently selected from N, O and S; wherein the fused ring system consists of a 6-membered ring fused with either a 5-membered ring or another 6-membered ring, either ring of which is attached to the rest of the compound via a carbon atom; wherein the ring of the fused ring system attached to the rest of the compound via the carbon atom contains at least one of the heteroatoms; and wherein the fused ring system is:
(i) optionally substituted with 1 or 2 substituents each of which is independently a —C₁₋₄ alkyl; and
(ii) optionally substituted with aryl or —C₁₋₄ alkyl-aryl;
HetC is a 4- to 7-membered saturated heterocyclic ring containing at least one carbon atom and a total of from 1 to 4 heteroatoms independently selected from 1 to 4 N atoms, from 0 to 2 O atoms, and from 0 to 2 S atoms, wherein any ring S atom is optionally oxidized to SO or SO₂, and wherein the heterocyclic ring is optionally fused with a benzene ring, and wherein the heterocyclic ring is attached to the rest of the compound via a N atom in the ring, and wherein the heterocyclic ring is:
(i) optionally substituted with 1 or 2 substituents each of which is independently a —C₁₋₄ alkyl, —C₁₋₄ alkyl-N(R^{a})R^{b}, or —C(═O)ORₐ; and
(ii) optionally substituted with aryl, —C₁₋₄ alkyl-aryl, HetD, or —C₁₋₄ alkyl-HetD; wherein HetD is (i) a 5- or 6-membered heteroaromatic ring containing from 1 to 4 heteroatoms independently selected from N, O and S or (ii) a 4- to 7-membered saturated heterocyclic ring containing at least one carbon atom and from 1 to 4 heteroatoms independently selected from N, O and S;
aryl is phenyl or naphthyl;
R^{a} is H or C₁₋₄ alkyl; and
R^{b} is H or C₁₋₄ alkyl.

12. A compound according to claim 11, or a pharmaceutically acceptable salt thereof, wherein R¹ is:
(1) —CF₃,
(2) —C(═O)—CH₃,
(3) —CO₂H,
(4) —C(═O)OCH₃,
(5) —C(═O)—NH(CH₃),
(6) —C(═O)—N(CH₃)₂,
(7) —C(═O)—NH(CH₂CH₃),
(8) —C(═O)—N(CH₂CH₃)₂,
(9) —C(═O)—NH(CH(CH₃)₂),
(10) —C(═O)—NH—CH₂-phenyl,
(11) —C(═O)—N(CH₃)—CH₂-phenyl,
(12) —HetB,
(13) —C(═O)—NH—CH₂—HetB,
(14) —C(═O)—N(CH₃)—CH₂—HetB, or
(15) —C(═O)—HetC.

13. A pharmaceutical composition comprising an effective amount of a compound according to claim 1, or a pharmaceutically acceptable salt thereof, and a pharmaceutically acceptable carrier.

14. A method of inhibiting HIV integrase in a subject in need thereof which comprises administering to the subject an effective amount of the compound according to claim 1, or a pharmaceutically acceptable salt thereof.

15. A method for preventing or treating infection by HIV or for preventing, treating or delaying the onset of AIDS in a subject in need thereof which comprises administering to the subject an effective amount of the compound according claim 1, or a pharmaceutically acceptable salt thereof.

16. A pharmaceutical combination which is (i) a compound according to claim 1, or a pharmaceutically acceptable salt thereof, and (ii) an HIV infection/AIDS antiviral agent selected from the group consisting of HIV protease inhibitors, non-nucleoside HIV reverse transcriptase inhibitors and nucleoside HIV reverse transcriptase inhibitors; wherein the compound of (i) or its pharmaceutically acceptable salt and the HIV infection/AIDS antiviral agent of (ii) are each employed in an amount that renders the combination effective for inhibiting HIV integrase, for treating or preventing infection by HIV, or for preventing, treating or delaying the onset of AIDS.

17. (canceled)

18. (canceled)
